SEQUENCE LISTING (16 sequences)

SEQ ID NO: 1 - protein, 183 aa, Homo sapiens
MSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGIC

SEQ ID NO: 2 - protein, 331 aa, Homo sapiens
MSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVYPYDVPDYA

SEQ ID NO: 3 - protein, 277 aa, Homo sapiens
MELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICK

SEQ ID NO: 4 - protein, 460 aa, Homo sapiens
MSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICMELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICK

SEQ ID NO: 5 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCMVEGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, Mouse
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 7 - protein, 20 aa, Artificial Sequence
  synthetic construct
MYRMQLLSCIALSLALVTNS

SEQ ID NO: 8 - protein, 435 aa, Homo sapiens
MYRMQLLSCIALSLALVTNSAIAMSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCMVEGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 583 aa, Homo sapiens
MYRMQLLSCIALSLALVTNSAIAMSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVYPYDVPDYALEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCMVEGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 529 aa, Homo sapiens
MYRMQLLSCIALSLALVTNSAIAMELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKLEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCMVEGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 440 aa, Artificial Sequence
  synthetic construct
MYRMQLLSCIALSLALVTNSAIAMSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 12 - protein, 588 aa, Artificial Sequence
  synthetic construct
MYRMQLLSCIALSLALVTNSAIAMSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVYPYDVPDYALEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 13 - protein, 534 aa, Artificial Sequence
  synthetic construct
MYRMQLLSCIALSLALVTNSAIAMELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 14 - DNA, 20 bp, Artificial Sequence
  synthetic construct
cctgcgactccttgacgttg

SEQ ID NO: 15 - DNA, 21 bp, Artificial Sequence
  synthetic construct
agcggtgaaagtggtttggtt

SEQ ID NO: 16 - DNA, 6 bp, Artificial Sequence
  synthetic construct
ttaggg

(19) United States
(12) Patent Application Publication
(10) Pub. No.: US 2023/0121867 A1
(43) Pub. Date: Apr. 20, 2023

(54) COMPOSITIONS AND METHODS FOR TREATING DISEASES AND CONDITIONS BY DEPLETION OF MITOCHONDRIAL OR GENOMIC DNA FROM CIRCULATION

(71) Applicant: Cedars-Sinai Medical Center, Los Angeles, CA (US)

(72) Inventors: Supriya BORAH, Los Angeles, CA (US); Neil BHOWMICK, Beverly Hills, CA (US); Subhash HALDAR, Los Angeles, CA (US)

(73) Assignee: Cedars-Sinai Medical Center, Los Angeles, CA (US)

(21) Appl. No.: 17/779,716

(22) PCT Filed: Nov. 25, 2020
(86) PCT No.: PCT/US2020/062330

§ 371 Date: May 25, 2022

Related U.S. Application Data
(60) Provisional application No. 62/940,457, filed on Nov. 26, 2019.

(51) Int. Cl. C07K 14/705; C07K 14/735; A61P 35/00

(52) U.S. Cl. CPC C07K 14/70596; A61P 35/00; C07K 14/70535; C07K 2319/30

(57) ABSTRACT

The present invention describes proteins that are capable to binding to mtDNA and/or gDNA and depleting circulating mtDNA and/or gDNA from a subject in need thereof. These proteins can be used to treat diseases and conditions such as cancer, cardiac infarction, and traumatic brain injury. These proteins can also be used to detect and measure circulating mtDNA and gDNA.

### CROSS-REFERENCE TO RELATED APPLICATIONS

This application includes a claim of priority under 35 U.S.C. §119(e) to U.S. Provisional Pat. Application No. 62/940,457, filed Nov. 26, 2019, the entirety of which is hereby incorporated by reference.

### FIELD OF INVENTION

This invention relates to the therapeutics for treating diseases and conditions such as cancer, cardiac infarction and traumatic brain injury.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Prostate cancer (PCa) is the second leading cause of cancer-related death of men in the United States. Since 2004, taxanes have become and remain an important mainstay of therapy for advanced PCa. Taxanes, inclusive of docetaxel, paclitaxel, and cabazataxel, hyperstabilize microtubules, to inhibit intracellular trafficking and signaling, cause mitotic arrest, and induce apoptotic cell death for numerous solid tumor types, inclusive of ovarian, breast, lung, head and neck, and prostate. Docetaxel was the first taxane to provide an overall survival benefit for men with metastatic, castrate-resistant prostate cancer. Its ability to even inhibit androgen signaling support its importance in PCa anticancer activity. Phase 2 studies have tested the use of taxanes, prior to androgen-targeted therapy failure, and demonstrated positive biochemical tumor response. Significantly, in the CHAARTED (Chemohormonal Therapy Versus Androgen Ablation Randomized Trial for Extensive Disease in Prostate Cancer) trial, the combined use of hormone therapy and docetaxel for castrate-sensitive PCa patients with high volume disease provided a significant survival advantage compared to castration therapy alone. In the STAMPEDE (Systemic Therapy in Advancing or Metastatic Prostate Cancer: Evaluation of Drug Efficacy) trial, docetaxel improved survival primarily in for men with metastatic castration-sensitive prostate cancer. Despite the importance of taxanes in the management of PCa, its utility is limited by toxicity and acquisition of chemo-resistance.

Accordingly, there is a need in the art for treatments that overcome these issues.

### SUMMARY OF THE INVENTION

The following embodiments and aspects thereof are described and illustrated in conjunction with compositions and methods which are meant to be exemplary and illustrative, not limiting in scope.

Various embodiments provide for a protein, comprising: a polypeptide that binds to mitochondrial DNA (mtDNA), genomic DNA (gDNA), or both; and a Fc fragment of IgG receptor gamma (FcgRIIb) or a fragment thereof.

In various embodiments, the polypeptide that binds to mtDNA, gDNA or both can comprise a fragment of DEC205 or a fragment of DEC205 with one or more amino acid deletions, additions or substitutions.

In various embodiments, the fragment of DEC205 can be a polypeptide at least 90% identical to at least one domain selected from the group consisting of Ricin B-type lectin domain, fibronectin type II lectin domain, and at least one C-type lectin domain. In various embodiments, the fragment of DEC205 can be a polypeptide at least 90% identical to at least two domains selected from the group consisting of Ricin B-type lectin domain, fibronectin type II lectin domain, and at least one C-type lectin domain. In various embodiments, the fragment of DEC205 can be a polypeptide at least 90% identical to at least three domains selected from the group consisting of Ricin B-type lectin domain, fibronectin type II lectin domain, and at least one C-type lectin domain. In various embodiments, the fragment of DEC205 can be a polypeptide at least 90% identical to Ricin B-type lectin domain, fibronectin type II lectin domain, or both. In various embodiments, the fragment of DEC205 can be a polypeptide at least 90% identical to Ricin B-type lectin domain and fibronectin type II lectin domain. In various embodiments, the fragment of DEC205 can be a polypeptide at least 90% identical to Ricin B-type lectin domain, fibronectin type II lectin domain, and at least one C-type lectin domain. In various embodiments, the fragment of DEC205 can be a polypeptide at least 90% identical to at least one C-type lectin domain. In various embodiments, the fragment of DEC205 can be a polypeptide at least 90% identical to at least two C-type lectin domains. In various embodiments, the fragment of DEC205 can comprise a polypeptide is at least 90% identical to a sequence selected from the group consisting of SEQ ID NO:1, SEQ ID NO:2, and SEQ ID NO:3. In various embodiments, the fragment of DEC205 can comprise a polypeptide that has a sequence selected from the group consisting of SEQ ID NO:1, SEQ ID NO:2, and SEQ ID NO:3. In various embodiments, the fragment of DEC205 can comprise a polypeptide is at least 90% identical to a sequence comprising SEQ ID NO:4. In various embodiments, the fragment of DEC205 can comprise a polypeptide having at least 168 consecutive amino acids of SEQ ID NO:4. In various embodiments, the fragment of DEC205 can comprise a polypeptide having 168 to 414 consecutive amino acids of SEQ ID NO:4. In various embodiments, the fragment of DEC205 can comprise a polypeptide having 183 to 368 consecutive amino acids of SEQ ID NO:4. In various embodiments, the fragment of DEC205 can comprise a polypeptide having 202 to 322 consecutive amino acids of SEQ ID NO:4. In various embodiments, the fragment of DEC205 can comprise a polypeptide having 220 to 276 consecutive amino acids of SEQ ID NO:4.

In various embodiments, the Fc fragment of IgG receptor gamma (FcgRIIb) comprises a human IgG1 Fc domain or a human IgG1 Fc domain with up to 22 amino acid additions, deletions, and/or substitutions. In various embodiments, the Fc fragment of IgG receptor gamma (FcgRIIb) or the fragment thereof can comprise at least 205 consecutive amino acids as set forth in SEQ ID NO:5. In various embodiments, the Fc fragment of IgG receptor gamma (FcgRIIb) or the fragment thereof can comprise a sequence with at least 90% sequence identity with SEQ ID NO:5. In various embodiments, the Fc fragment of IgG receptor gamma (FcgRIIb) can comprise a polypeptide having the sequence as set forth in SEQ ID NO:5.

In various embodiments, the Fc fragment of IgG receptor gamma (FcgRIIb) can be a mouse IgG1 Fc domain, or a mouse IgG1 Fc domain with up to 21 amino acid additions, deletions, and/or substitutions. In various embodiments, the Fc fragment of IgG receptor gamma (FcgRIIb) or the fragment thereof can comprise at least 209 consecutive amino acids as set forth in SEQ ID NO:6. In various embodiments, the Fc fragment of IgG receptor gamma (FcgRIIb) or the fragment thereof can comprise a sequence with at least 90% sequence identity with SEQ ID NO:6. In various embodiments, the Fc fragment of IgG receptor gamma (FcgRIIb) can comprise a polypeptide having the sequence as set forth in SEQ ID NO:6.

In various embodiments, the protein can further comprise a signal sequence, a linker, or both. In various embodiments, the signal sequence can comprise the amino acids as set forth in SEQ ID NO:7.

In various embodiments, the protein can be selected from a protein having the sequence as set forth in any one of amino acids 24-435 of SEQ ID NO:8, amino acids 24-583 of SEQ ID NO:9, amino acids 24-529 of SEQ ID NO:10, amino acids 24-440 of SEQ ID NO:11, amino acids 24-588 of SEQ ID NO:12, or amino acids 24-534 of SEQ ID NO: 13.

In various embodiments, the protein can be selected from a protein comprising the sequence of SEQ ID NO:1 and SEQ ID NO:5; or SEQ ID NO:2 and SEQ ID NO:5; or SEQ ID NO:3 and SEQ ID NO:5; or SEQ ID NO:1 and SEQ ID NO:6; or SEQ ID NO:2 and SEQ ID NO:6; or SEQ ID NO:3 and SEQ ID NO:6.

In various embodiments, the protein can be selected from a protein having the sequence as set forth in any one of SEQ ID NO:8, SEQ ID NO:9, SEQ ID NO:10, SEQ ID NO: 11, SEQ ID NO:12, or SEQ ID NO: 13.

In various embodiments, the polypeptide that binds to mtDNA, gDNA or both comprises a fragment of toll-like receptor 9 (TLR9) or a fragment of TLR9 with one or more amino acid deletions, additions or substitutions.

In various embodiments, the protein can further comprise an Fc region of an antibody or a fragment thereof.

In various embodiments, the protein can be capable of depleting circulating mtDNA. In various embodiments, the protein can be capable of depleting circulating genomic DNA (gDNA).

Various embodiments of the present invention provide for a nucleic acid encoding any one of the proteins of the present invention as described herein.

Various embodiments of the present invention provide for a cell producing any one of the proteins of the present invention.

Various embodiments of the present invention provide for a cell comprising any one of the nucleic acids of the present invention.

In various embodiments, the cell can be a bacterial cell, a Chinese hamster ovarian cell (CHO) or a baby hamster kidney cell (BHK). In various embodiments, the bacterial cell is Bacillus subtilis or Lactococus lactis.

Various embodiments of the present invention provide for a combination, comprising: any one of the proteins of the present invention; and a therapeutic agent.

In various embodiments, the therapeutic agent can be selected from the group consisting of an anti-tumor agent, a chemotherapeutic agent, an androgen ablating agent, a cardiac infarction treatment agent, a traumatic brain injury treatment agent, and combinations thereof. In various embodiments, the therapeutic agent can be a taxane, anthracycline, or a platinum based antineoplastic drug. In various embodiments, the therapeutic agent can be docetaxel, paclitaxel, cabazataxel, doxorubicin, epirubicin, idarubicin, valrubicin, cisplatin, oxaliplatin, carboplatin, irinotecan, or fluorouracil (5 FU). In various embodiments, the therapeutic agent can be an androgen receptor antagonist, an androgen synthesis inhibitor, or an anti-gonadotropin. In various embodiments, the therapeutic agent can be selected from the group consisting of bicalutamide, enzalutamide, apalutamide, flutamide, nilutamide, darolutamide, cyproterone acetate, megestrol acetate, chlormadinone acetate, spironolactone, oxendolone, ketoconazole, abiraterone acetate, seviteronel, aminoglutethimide, finasteride, dutasteride, epristeride, alfatradiol, saw palmetto extract, leuprorelin, cetorelix and combinations thereof. In various embodiments, the therapeutic agent can be aspirin, a thrombolytic agent, heparin, an antiplatelet agent, nitroglycerin, a beta blocker, an ACE inhibitor, a statin, and combinations thereof. In various embodiments, the therapeutic agent can be a diuretic, an anti-seizure drug, a coma-inducing drug, or combinations thereof.

Various embodiments of the present invention provide for a device, comprising: at least one inlet; at least one outlet; at least one chamber comprising a solid substrate; and any one of the proteins of the present invention immobilized on the solid substrate.

In various embodiments, the device can be a microfluidic device.

In various embodiments, the solid substrate can be dextran beads or sepharose beads.

Various embodiments of the present invention provide for a device, comprising any one of the proteins of the present invention immobilized onto a solid substrate.

In various embodiments, the solid substrate can be a multi-well plate. In various embodiments, the solid substrate can be a bead.

In various embodiments, the protein can be further conjugated or immobilized to a conductive substrate to produce a detectable signal upon binding to mtDNA, gDNA, or both.

In various embodiments, the conductive substrate can be gold, silver, platinum, iridium, or copper.

In various embodiments, the protein can be further conjugated or immobilized to silicone.

Various embodiments of the present invention provide for a method of reducing circulating mitochondrial DNA (mtDNA), genomic DNA (gDNA), or both in a mammalian subject, comprising: administering any one of the proteins of the present invention, or administering any one of the combinations of the present invention to the mammalian subject, or removing circulating mtDNA, gDNA, or both from the mammalian subject’s blood, or administering any one of the bacterial cells of the present invention to the mammalian subject.

In various embodiments, the mammalian subject can have or can be suspected to have a disease or condition caused by or related to elevated levels of circulating mitochondrial DNA (mtDNA), genomic DNA (gDNA), or both.

In various embodiments, the disease or condition can be selected from the group consisting of a tumor, cancer, cardiac infarct, cardiac disease, physical trauma, traumatic brain injury, infection, stroke, inflammation, autoimmune disease, cachexia, and lupus.

In various embodiments, the cancer can be a solid tumor cancer. In various embodiments, the cancer can be prostate cancer or breast cancer.

In various embodiments, removing circulating mtDNA from the mammalian subject’s blood can comprise passing the subject’s blood through any one of the devices of the present invention.

Various embodiments of the present invention provide for a method of measuring circulating mitochondrial DNA (mtDNA), genomic DNA, or both comprising: obtaining a biological sample; contacting any one of the proteins of the present invention to the biological sample; detecting the binding of the protein to the mtDNA, gDNA, or both; and quantifying the amount of protein-mtDNA binding conjugate, protein-gDNA binding conjugate, or both.

In various embodiments, the protein can further comprise a label to produce a detectable signal. In various embodiments, the detectable signal can be colorimetric, fluorescence, or luminescence.

In various embodiments, the protein can be contacted to the biological sample using any one of the devices of the present invention.

In various embodiments, the device can comprise a conductive substrate and the protein is conjugated or immobilized to the conductive substrate to produce a detectable signal upon binding to mtDNA, gDNA or both, wherein the detectable signal is impedance, resistance, change in current, or change in electrochemical impedance spectrum, and wherein the conductive substrate is selected from the group consisting of gold, silver, platinum, iridium, copper.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1, panels A-H, depicts Activation of TLR9 and C3a by mtDNA. A Mitochondrial DNA was measured from conditioned medium (CM) of prostatic epithelia after 48 hrs of incubation (n=3). B Protein expression in CAF treated with LNCaP-CM was visualized by western blot. C DEC205 expressions was measured in NAF and CAF treated with LNCaP-CM by western blot. D DEC205 was immunoprecipitated, crosslinked, and subjected to mtDNA PCR amplification for MT-CO2 following CAF incubation with LNCaP-CM. CAF cell lysate prior to immunoprecipitation or IgG immunoprecipitant was used as total input and negative controls, respectively. E mRNA expression profiles of the NF-κB signaling targets in CAF incubated with LNCaP-CM were compared to control-CM in the heat-map (n=4). F Volcano plot showing distribution of differential mRNA expression level in CAF and CAF incubated with LNCaP-CM. While only secreted proteins were illustrated in the heatmap, all 84 NF-XκB target genes were represented in the volcano plot. G TLR9 and anaphylatoxin C3a protein expression was visualized in CAF incubated with LNCaP-CM incubated with or without DNase1 treatment. DNase activity was heat inactivated after 10 min. H LNCaP-CM contain mtDNA that binds DEC205 for internalization in CAF cells for subsequent TLR9 signaling and anaphylatoxin C3a expression. *P < 0.05, **P < 0.01.

FIG. 2, panels A-G, depicts Mechanism of C3a generation by CAF. A TLR9 signaling was tested in mouse prostatic fibroblasts from cultured wild type (WT) or TLR9-knockout (TLR9^{-/-}) mice treated with CpG-ODN or LNCaP-CM in the presence and absence of DNase1 treatment. B Secreted C3a was measured by ELISA from CAF and NAF conditioned media following treatment with CpG-ODN, LNCaP-CM or TRAMPC2-CM (n=3). C Flow cytometry was used to quantitate intracellular reactive oxygen by DCFDA fluorescence in CAF incubated with control, CpG-ODN or LNCaP-CM as determined by quantitating DCFDA⁺ cells absent of 7AAD staining. D Green DCFDA fluorescence was cytoplasmically localized with DAPI nuclear counter stain by fluorescence microscopy. The scale bar represents 16 µm. E Catalase activity was quantitated in CAF following incubation with fresh media (control), CpG-ODN or LNCaP-CM. F Protein expression of complement C3 and anaphylatoxin C3a in CAF was western blotted. The CAF were incubated with either CpG-ODN in the presence and absence of catalase inhibitor, 3-Amino-1,2,4-triazole (3AT) or LNCaP-CM in the presence or absence of reactive oxygen inhibitor, n-acetyl cysteine (NAC). G MtDNA from in LNCaP-CM binds DEC205 for internalization in CAF cells for subsequent TLR9 signaling. LNCaP-CM inhibited catalase activity allow ROS production makes C3a in the CAF. *P < 0.05, **P < 0.01, ***P < 0.001, and ns - not significant.

FIG. 3, panels A-E, depicts Role of C3a in PCa progression. A PCa cell lines incubated in the absence and presence of C3a receptor agonist peptide (48 hr) was western blotted for cell survival and proliferation protein expression. B C57BL/6 mice were allografted with tissue recombinants of luciferase-expressing TRAMPC2 with wild type (wt) or Tlr9^{-/-}fibroblasts. The mice were treated with saline or TLR9 antagonist, SB290157. Luciferase bioluminescence was used to image tumor progression. C Mean tumor volume (mm³) and standard deviation (S.D.) for each treatment condition are depicted (n=8). D H&E and immunohistochemistry for phosphorylated-AKT, phosphorylated-histone-H3 and TUNEL staining of the tumor tissues were performed and quantitated. The corresponding graphs illustrate the mean and S.D. expression of the staining (n=4). *P < 0.05; **P < 0.01. The scale bar represents 10 µm. E FACS analysis of the tumor tissues demonstrated C3a antagonist and TLR9-knockout fibroblasts had similar CD3⁺ T cell infiltration, however their activation state as determined by CD8⁺/CD69⁺ expression differed significantly, compared to control.

FIG. 4, panels A-G, depicts Docetaxel promotes mtDNA release from PCa cells and paracrine TLR9 signaling contribute to therapeutic resistance. A Plasma levels of mtDNA was quantified from PCa patients before and after docetaxel treatment (n=9). B MtDNA content of plasma from mice treated with docetaxel was quantitated (n=3). Data represent the mean ± S.D., *P<0.05. C MtDNA secreted by PCa cell lines treated with docetaxel was elevated in a dose-dependent manner (n=3). Significance was determined by repeated measures ANOVA. D LNCaP cells treated with vehicle or docetaxel was subjected to subcellular fractionation. The mitochondrial localization of mitophagy markers, p62, Pink1, and Beclin, was confirmed by the co-expression of Tom20. The cytoplasmic fraction was confirmed by the expression of Rho A. E MtDNA secretion resulting from ER-stress, as indicated by CHOP expression, was evident in LNCaP and PC3 cells treated with docetaxel. F Treatment of a three-dimensional co-culture model of PC3 and CAF cells with docetaxel and TLR9 antagonist, SB290157, supported differential epithelial proliferation as determined by quantitating EPCaM⁺/Ki-67⁺ cells by FACS analysis (n=3). G Synergistic cooperativity was identified in PC3 cell viability measured by MTT assay following treatment with docetaxel and SB290157 through the Chou-Talalay method (n=4). Values below the confidence interval (CI) of 1 (line) are considered to indicate a synergistic combination.

FIG. 5, panels A-C, depicts Synergistic effect of docetaxel and SB 290157 inhibit tumor growth. A Subcutaneous xenografts of PC3 and CAF tumor volumes were longitudinally measured. When tumor average volume reached 80 mm³ mice were treated with vehicle or docetaxel in the presence or absence of SB290157 for 20 days (n = 4). Representative images show each group of mice (inset). B Immunoblots of the tumor tissues for the respective treatments are demonstrated (n=3). C Immunolocalization ofphosphorylated-TAK1, complement C3, phosphorylated-AKT, phosphorylated-histoneH3 and TUNEL expression in tumor tissues (brown) was counterstained with hematoxylin (blue). The corresponding bar graph illustrate the mean and S.D. expression of the respective staining (n=5). Data represent the mean ± S.D. by one-way ANOVA (*P <0.05; **P <0.01). The scale bar represents 10 µm.

FIG. 6 depicts Schematic illustration of the PCa epithelia and CAF reciprocal interaction. PCa cells generate mtDNA that can bind endocytic DEC205 on the cell surface of CAF. TLR9 signaling downstream of epithelial-derived mtDNA results in NF-κB mediated C3 expression. The accumulation of ROS in CAF enables C3a maturation and paracrine signaling with PCa cells that enables cell survival and proliferation. Docetaxel treatment of PCa cells potentiate ER stress and mitophagy for the expanded secretion of mtDNA in perpetuating the further C3a expression by CAF.

FIG. 7, panels A-F, depicts A Relative mRNA expression of TLR9 was measured in the presence and absence of BPH1 conditioned medium (CM) and LNCaP-CM in cultured NAF or CAF. B Measurement of telomere and mitochondrial DNA concentration from conditioned medium of cultured human prostate cancer cells. C Protein expression of caspase1 and IL-1β from cultured CAF treated with LNCaP-CM. Lower molecular weight cleaved-caspase1 and mature active-IL1β induced by LNCaP-CM was limited by DNase1 treatment and subsequent heat inactivation. s-actin expression was used as a loading control. D LNCaP-CM induced TLR9 mRNA expression by cultured CAF was limited by DNase1, but not sonication of the conditioned media. E Inhibition of dynamin-mediated exosome secretion with increasing doses of dynasore had no effect on the secretion of mtDNA by LNCaP cells. F Protein expression of HMGB1 and HMGA2 by NAF and CAF was subjected to western blotting following LNCaP-CM treatment. *P < 0.05, **P < 0.01, ***P < 0.001.

FIG. 8, panels A-C, depicts A C3a receptor (C3a-R) mRNA expression was similarly expressed by cultured LNCaP, PC3 and TrampC2 cells. B Western blot for the expression of DEC205, TLR9, HMGB1, and C3a was performed on the indicated PCa epithelial cell lines. C Proliferation of LNCaP, PC3 and TrampC2 cells was quantitated by measuring Ki-67 through FACS analysis following treatment with C3aR agonist or scrambled peptides for 48 h, (n = 3).

FIG. 9, panels A-C, depicts A nn interaction index and confidence intervals were calculated by Chou-Talalay method for determining a synergistic relationship between SB290157 and docetaxel treatment at the indicated treatment concentrations of PC3 cells by the MTT viability assay. B Mice harboring subcutaneous xenografts of PC3/CAF tumors were weighed throughout the saline, docetaxel alone, or combination with SB290157 treatment course. Data represent the mean ± S.D. among groups by oneway ANOVA (ns - not significant). C H&E images of resulting tumors from each treatment group of subcutaneous xenografted mice.

FIG. 10 depicts the extracellular domain of DEC205 contain multiple lectin domains: Ricin B-type lectin, fibronectin type II lectin domain, and ten C-type lectin domains. Three antibody Fc domain conjugates were generated containing the Ricin B-type and fibronectin type II domains (RF-Fc), the Ricin B-type, fibronectin type II domains and C-type lectin (RFL-Fc), and two C-type lectin domains.

FIG. 11 depicts three DEC205 fragments, RF, RFL, and 2L conjugated to the IgG1 Fc domain. Conditioned media from CHO-K1 cells stably expressing the respective constructs were subjected to protein G affinity purification, run on 10% acrylamide gel, and visualized by Coomassie staining.

FIG. 12 depicts ELISA testing the binding of RF-Fc and RFL-Fc of (A) mtDNA and (B) gDNA. RF-Fc binds mtDNA 2-fold over gDNA. RFL-Fc has similar capacity to bind mtDNA and gDNA. Absorbance was taken at 570 nm. OD values are normalized for their respective Fc concentrations. **P < 0.01, ***P < 0.001, ****P < 0.0001.

FIG. 13 depicts The basis for docetaxel resistance potentiated by mtDNA is the expression of complement C3 by cancer associated fibroblastic cells (PNAS 2020 11:8515). When conditioned media from prostate cancer cells (PC3) were incubated with cancer associated fibroblastic cells C3 expression was significantly downregulated by the depletion of mtDNA using RF-Fc. **P < 0.01.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor NY, 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. Sep;23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 9%, 8%, 7%, 6%, 5%, 4%, 3%, 2%, or 1% of that referenced numeric indication, if specifically provided for in the claims.

The term “biological sample” as used herein denotes a sample taken or isolated from a biological organism. Exemplary biological samples include, but are not limited to body fluids, whole blood, plasma, serum, stool, intestinal fluids or aspirate, and stomach fluids or aspirate, cerebral spinal fluid (CSF), urine, sweat, saliva, tears, pulmonary secretions, breast aspirate, prostate fluid, seminal fluid, cervical scraping, amniotic fluid, intraocular fluid, mucous, and moisture in breath. In various embodiments, the biological sample may be whole blood. In various embodiments, the biological sample may be serum. In various embodiments, the biological sample may be plasma. The term also includes a mixture of the above-mentioned samples.

As used herein, the term “label” refers to a composition capable of producing a detectable signal indicative of the presence of a target. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means needed for the methods and devices described herein. For example, the peptides can be labeled with a detectable tag which can be detected using an antibody specific to the label.

Exemplary fluorescent labeling reagents include, but are not limited to, Hydroxycoumarin, Succinimidyl ester, Aminocoumarin, Methoxycoumarin, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5 or Cy7.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program’s alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Described herein, we examined the role of the PCa microenvironment in docetaxel chemo-resistance. Stromal-epithelial interactions define tumor initiation, progression, and therapeutic resistance. In the prostate tumor microenvironment, stromal fibroblasts co-evolve with the cancer epithelia in a reciprocal relationship. The central role of cancer-associated fibroblasts (CAF) was recognized when their absence was found to result in reduced tumor volumes. CAF have been shown to produce paracrine growth factors, proteolytic enzymes and components of the extracellular matrix, presumably in response to cues from tumor cells. In fact, CAF derived from breast cancer patients treated with docetaxel were found to secrete greater tumor supportive factors compared to the CAF derived from treatment-naive patients. However, the mechanisms regulating this crosstalk occurs are not well elucidated in the context of chemotherapy.

There is a large body of evidence describing the role of mitochondrial DNA (mtDNA) in PCa. Proteins in mitochondrial complexes I, III, IV, and V involved in oxidative phosphorylation are encoded by mtDNA. Mutations found in mtDNA increase tumorigenicity in PCa and deregulated mitochondrial metabolism is known to promote prostate carcinogenesis. PCa cells have greater mitochondrial content than benign prostate epithelium and alterations in mtDNA copy number may reflect disruption of the normal prostate glandular architecture. Furthermore, mtDNA instability is a hallmark of human cancers. PCa patients are found to have measurable concentrations of mtDNA in serum. Further described herein, we tested whether secreted mtDNA functions as a mediator of epithelia-CAF crosstalk. We reasoned the mtDNA could potentially signal adjacent cells through pattern recognition receptors, such as toll-like receptor 9 (TLR9). We identified a stromal-epithelia reciprocal signaling cascade initiated by docetaxel involving the TLR9 signaling in CAF and downstream paracrine response by PCa epithelia contributing to taxane therapy resistance.

As further described herein mitochondrial DNA (mtDNA) is expelled by cells undergoing stress, often in the form of endoplasmic reticulum stress (ER stress), in response to stimuli such as chemotherapy, androgen ablation treatment, cardiac infarction, and traumatic brain injury. In each case, mtDNA released by the cells can be perceived by neighboring or potentially distant cells through a specialized receptor, Toll like receptor 9 (TLR9). TLR9 signaling can promote an inflammatory cascade that causes recruitment of inflammatory cells, promotion of tumor cell growth, and cause longer term ramifications such as increased risk for a cardiac event or dementia associated diseases of the brain. Therefore, ridding the body of mtDNA so that TLR9 is not activated, can prevent the downstream inflammatory signals that contribute to multiple pathologies. The inventors' found mtDNA’s impact on tumor expansion and therapeutic resistance. The inventors further designed methods by which mtDNA is depleted from circulation by an engineered antibody inclusive of the application TLR9 mtDNA-binding domain and DEC205 as a method of capturing mtDNA for excretion by targeting to the hepatic vasculature.

Inflammation suppressors such as steroids and non-steroidal analgesics are available. But, there are no inhibitors that remove the initiator of such inflammatory cascades associated with mtDNA secretion. We have designed methods of capturing mtDNA from circulation by the use of an antibody variable region mimicking TLR9 or DEC205.

The work provides an advance in functionally defining the cross-talk of tumor epithelia with cancer-associated fibroblastic cells contributing to tumor progression and therapeutic resistance. Independent of protein-based signaling molecules, prostate cancer cells secreted mitochondrial DNA to induce associated fibroblasts to generate anaphylatoxin C3a to support tumor progression in a positive feed-back loop. Interestingly, the standard of care chemotherapy, docetaxel, used to treat castrate resistant prostate cancer was found to further potentiate this novel paracrine signaling axis to mediate therapeutic resistance. Blocking anaphylatoxin C3a signaling cooperatively sensitized prostate cancer tumors to docetaxel. We revealed that docetaxel resistance is not a cancer cell-autonomous phenomena and targeting an immune modulator derived from cancer associated fibroblasts can limit the expansion of docetaxel-resistant tumors.

Our data show reciprocal paracrine signaling between PCa and associated fibroblasts promote cancer progression and facilitates docetaxel resistance. We hypothesized mtDNA could be the paracrine signaling molecule generated by PCa cells (FIG. 6). The docetaxel-induced mtDNA secretion from PCa cells into the tumor microenvironment was significantly greater than the basal levels of mtDNA secreted by PCa cells. Accordingly, prostate tumors in both murine models and men harboring prostate tumors demonstrated elevated circulating mtDNA when treated with docetaxel. For subsequent CAF signaling, the mtDNA required entry into the cytoplasm for TLR9 activation. Based on the previous demonstration of DEC205 capture of CpG in dendritic cells (24), a similar scenario was explored for the prostatic CAF. Instead of unmethylated bacterial DNA, we demonstrated that in fact, DEC105 could directly bind mtDNA on CAF cells for classic pattern recognition receptor, TLR9, activation of TAK1 and NF-κB (37). TLR9 was identified to be essential for complement C3 expression by CAF in response to mtDNA, but the accumulation of reactive oxygen resulting from PCa-CM contributed to C3 cleavage and anaphylatoxin C3a generation. Released C3a in the tumor microenvironment increased proliferation of cancer cells and potentiated resistance to docetaxel treatment.

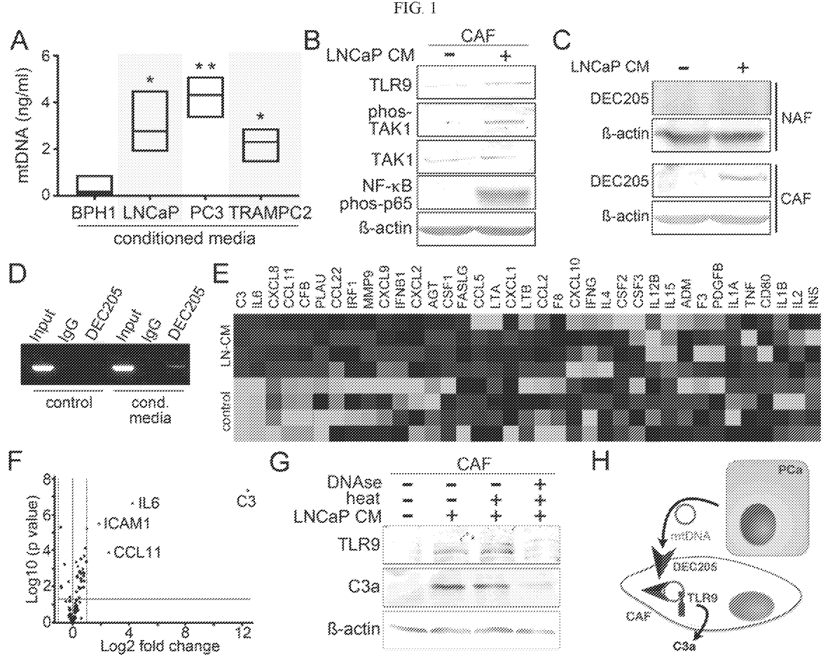

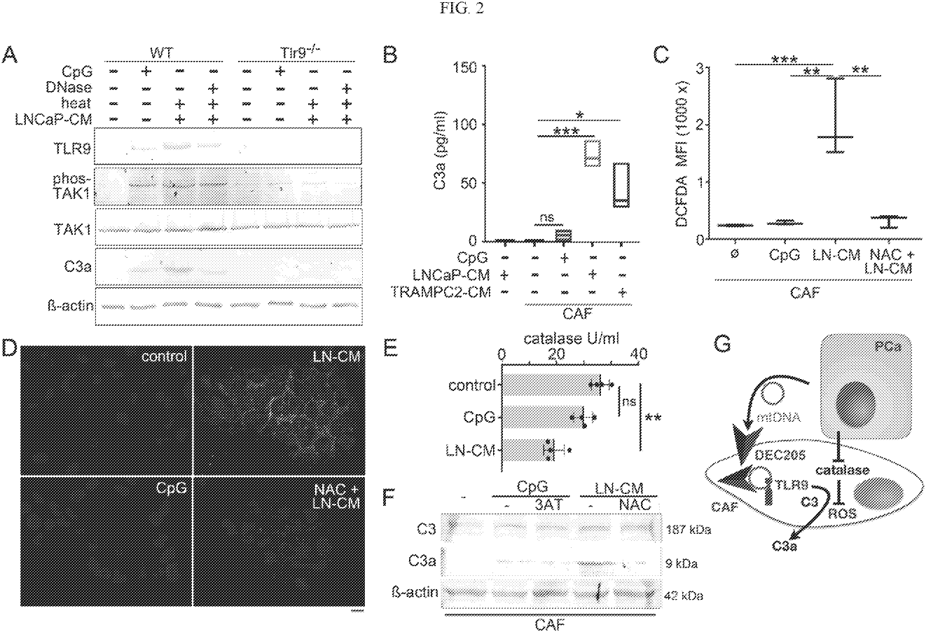

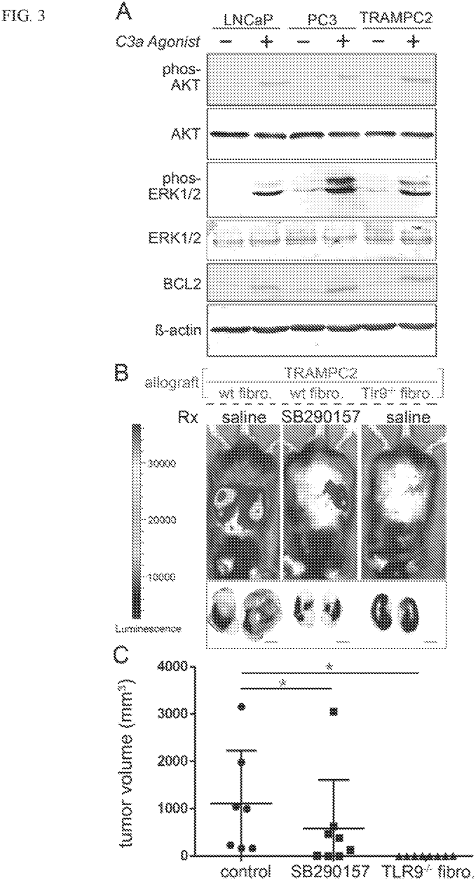

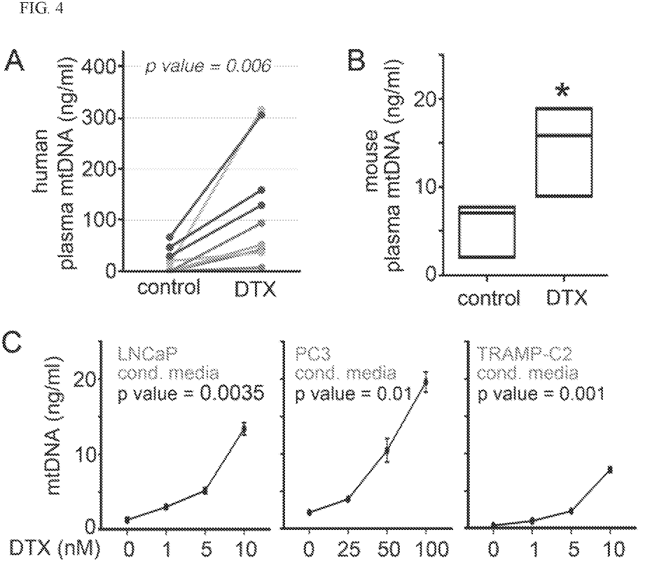

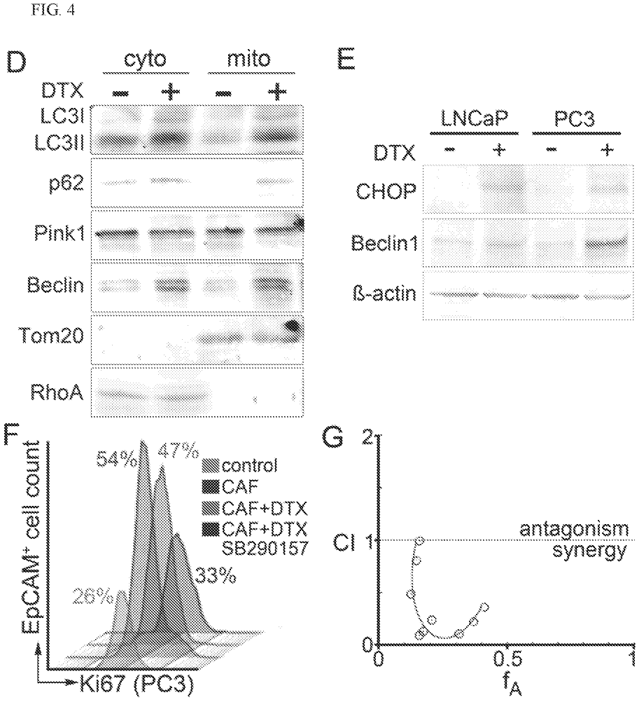

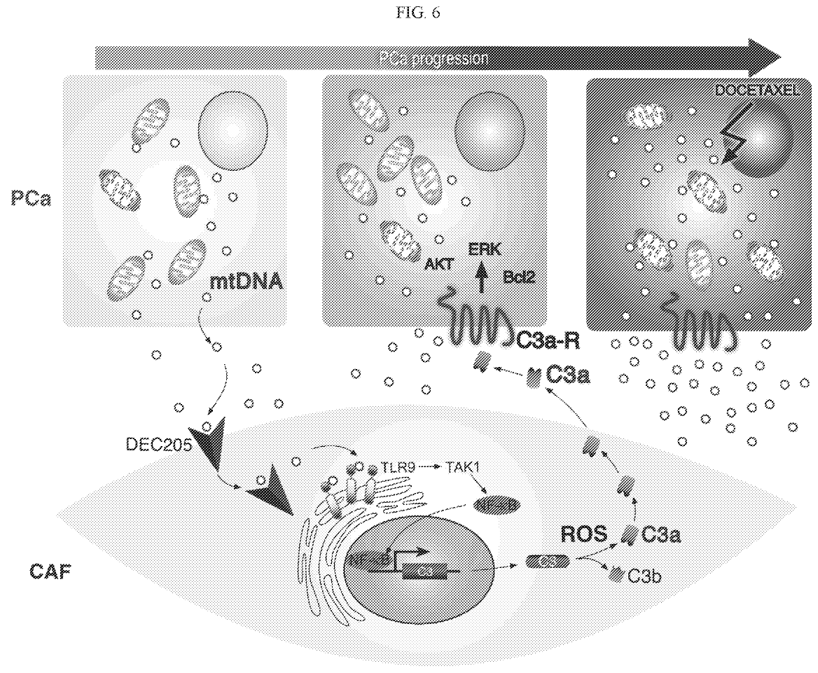

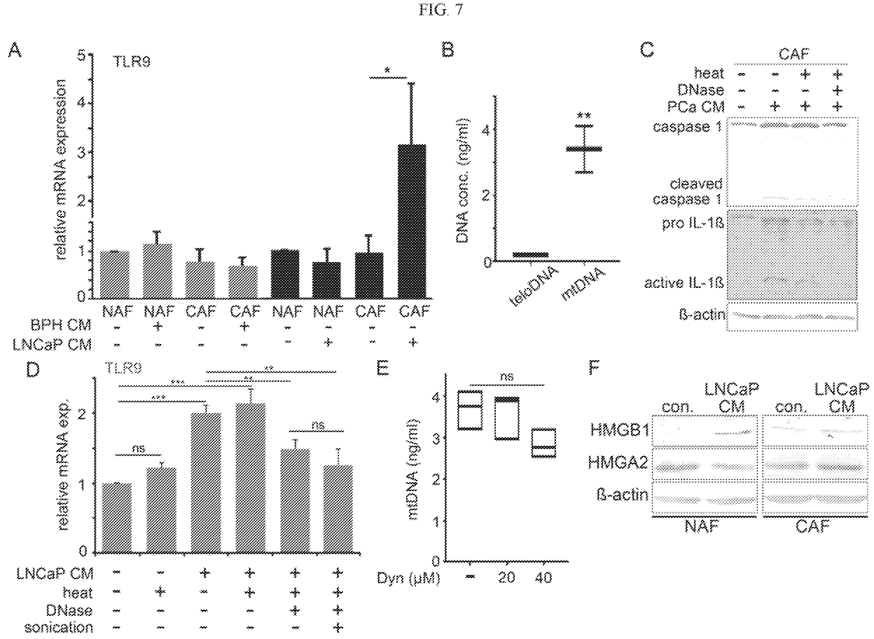

It is apparent that PCa-induced paracrine NF-κB activation in CAF dramatically potentiated complement C3 expression (>12 log-fold FIG. 1). There is a well-described immune defense for bacterial pathogens that include Toll-like receptor-mediated complement expression and generation of anaphylatoxin. However, the novel mechanism of TLR9 induction by PCa-derived mtDNA paracrine signal transduction mechanism in CAF cells was not observed in NAF cells (FIG. 1). Cell-free circulating mtDNA release in plasma at low levels under cellular stress is reported, in instances of cancer, trauma, infections, stroke, autoimmune, metabolic and rheumatic diseases. While activated T cells can signal dendritic cells through exosome-based delivery of mtDNA, this was likely not the means of paracrine communication between PCa and CAF. Dynamin inhibition or sonication of the PCa-CM had little effect on TLR9 expression/activity by CAF (FIG. 7). The especially low levels of telomeric DNA secreted by the PCa is noteworthy as it is a known to inhibit TLR9 signaling. Uniquely, DEC205 was expressed by CAF in the context of PCa-CM for endocytic delivery of mtDNA and TLR9 activation. This is the first time PCR amplification of the mitochondrial MT-CO2 gene following immunoprecipitation of DEC205 has been reported. Docetaxel potentiated PCa release of mtDNA by over 5-fold (FIGS. 1 and 4). Docetaxel treatment is reported to induce mTOR-mediated autophagy in prostate cancer cells. Treatment with chemotherapeutic drugs can cause ER-stress that enhance autophagic efflux from cells. Our identification of the combined ER-stress with mitophagy revealed means for the secretion of non-degraded mtDNA from PCa cells (FIG. 2). Thus, the initiation of the fibroblastic inflammatory cascade can be attributed to tumor-derived mtDNA signaling and the expression of complement C3. However, the activation of the complement system in response to pathogens involves three major pathways: 1) the classical pathway, via antigen-antibody complexes, 2) the lectin pathway, via binding of pattern-recognizing mannose-binding lectins, and 3) the alternative pathway, via any permissive microbe surfaces. In all three complement activation pathways, the C3 convertase complex cleaves C3 molecules to form anaphylatoxins C3a. Yet another mechanism of C3 conversion identified in neutrophils involving hydrogen peroxide-related oxygen radicals, such as hypochlorite, was the mechanism considered for the stromal-epithelial signaling axis. We found that catalase inhibition in CAF by PCa cells to be essential for ROS accumulation and maturation of anaphylatoxin C3a from C3 (FIG. 2). These findings explained the absence of C3a in CpG-ODN treated CAF cells in spite of NF-κB activation. Tumor-stromal interaction via mtDNA resulting in C3a expression by prostate fibroblasts was dependent on TLR9 activation and ROS-mediated complement maturation.

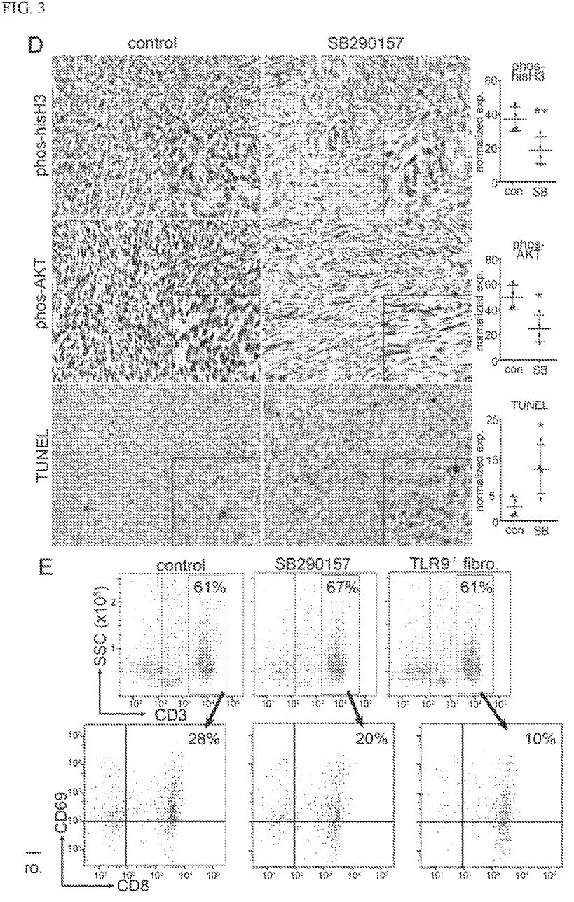

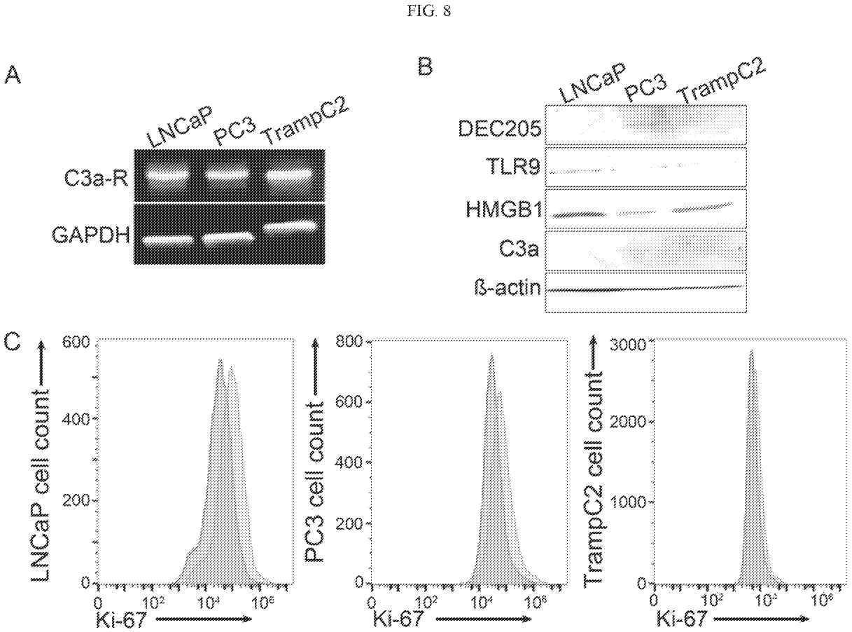

Our findings provide a paradigm in which the activation of complement was distinctly important for promoting tumor growth. There are studies that have reported a positive-growth effect of complement in cancer. The systemic level of complement proteins has an indirect effect on cancer growth by alteration of the immune response of host to the tumor. Wang et. al. showed that B16 melanoma growth was slower in C3 deficient mice than that in wild-type mice. Anaphylatoxin receptors signal through the PI3K/AKT pathway in cancer cells and the proliferative effect of C5aR and C3aR stimulation can be eliminated by AKT silencing. Here, we show PCa cells express receptors for C3a (FIG. 8). The CAF-derived C3a resulted in phosphorylated-AKT, phosphorylated-ERK1/2 and BCL2 upregulation in PCa epithelia (FIG. 3). Antagonizing the TLR9-C3a axis with SB290157 or stromal knockout of TLR9 significantly inhibited tumor expansion. We found a similar CD3⁺ T cells infiltration regardless of TLR9-C3a signaling alterations. However, CD8⁺/CD69⁺ activated cytotoxic T cells were significantly reduced by C3 antagonist and further diminished to nearly a third of control in tumors with TLR9-knockout fibroblasts. Accordingly, T cell-mediated tumor cell lysis was not the mechanism of the observed reduction in tumor size. Instead, C3a was likely acting directly on the tumor cells in a paracrine manner.

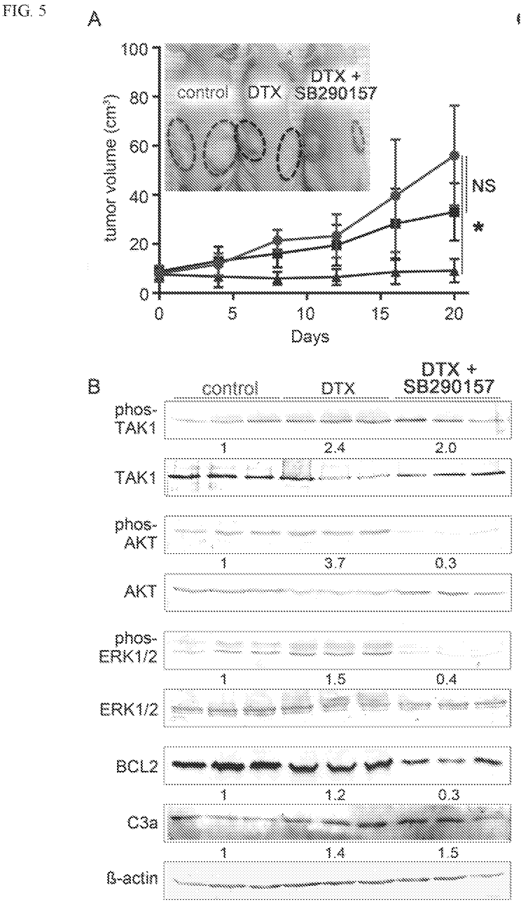

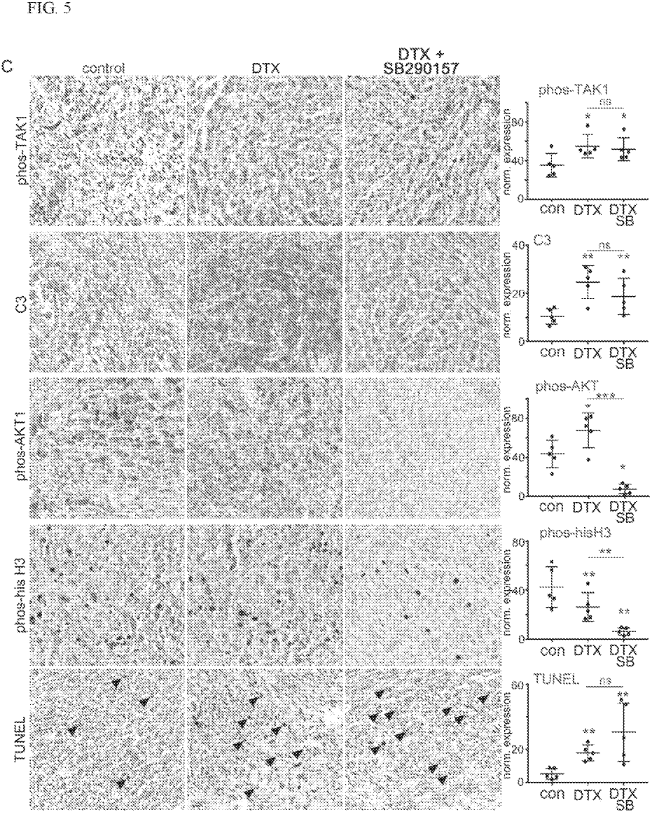

Docetaxel resistance is major clinical problem in many cancers including PCa. Activation of several survival signaling pathways can promote a resistant phenotype in response to docetaxel treatment. Docetaxel and complement signaling in PCa epithelia were observed to activate such survival signaling pathways (e.g. AKT and ERK with BCL2 expression) as well as autophagy (FIGS. 3 and 4). While autophagy in itself is a means of survival for neighboring cells through cellular catabolism, here we showed it also contributes to docetaxel-induced mtDNA secretion from PCa cells in its extension to mitophagy. The breakdown of mitochondria through mitophagy is inclusive of its DNA. However, in the context of ER stress, mitophagy can result in inadequate mtDNA degradation. Not surprisingly, docetaxel elicited ER stress on the PCa cells. The contribution of the CAF on PCa ER stress, while likely, was not explored. However, the CAF reciprocated the PCa-derived mtDNA signal by the TLR9-C3 paracrine axis to trigger a survival/proliferation signal in PCa cells. Investigation from mouse prostate tumor revealed docetaxel treatment enhanced C3a anaphylatoxin formation and mediated increased proliferation signaling. This proliferation signaling was reduced by blocking of C3a receptor (FIG. 4). Remarkably, antagonizing anaphylatoxin C3a signaling with SB290157 was able to sensitize an otherwise resistant PC3 cell line to docetaxel. The synergism of docetaxel and SB290157 allowed for reduced docetaxel doses to effectively limit tumor growth. Better understanding of the complement signaling axis in cancer cells is needed, as its implications can have a far-reaching impact on many cancer types currently treated with taxanes. Currently, docetaxel is in clinical trials in combination with immune checkpoint inhibition therapy to explore the potential for cooperative activity in stimulating infiltrating cytotoxic T cells. The induction of the stromal anaphylatoxin C3a mediated by docetaxel may contribute to immune-mediated cancer cell death (FIG. 3). We observed combining SB290157 with docetaxel did not result in greater apoptosis compared to docetaxel alone (FIG. 5). But, complement inhibition significantly limited proliferation and effectively reduced tumor size over that of docetaxel alone. Thus, one must weigh the benefits to immune-surveillance induced by docetaxel with the tumor intrinsic proliferative role of complement signaling.

Another implication of our results is that fibroblast response to taxane therapy is consequential to cancer epithelial therapeutic response. Circulating mtDNA has been reported to be a prognostic indicator for poor outcome for PCa patients. But, with the limited number of patients analyzed, we were unable to demonstrate a correlation with the level of mtDNA in circulation with length of docetaxel responsiveness. While the epithelial response to docetaxel can be uncoupled from that of the stromal fibroblasts, the stromal influence on therapeutic resistance is a result of a paracrine signaling axis, in this report, initiating from the PCa epithelia. Again, we cannot rule out the direct impact of docetaxel on CAF that could also influence epithelial viability. It should be noted that TLR-mediated NF-κB signaling is not a phenomenon limited to mammals. It was originally identified in Drosophila (Toll), with Toll9 involved in hematopoietic and digestive tract development. Although NF-κB regulation remains conserved the gene targets are species, tissue, and cell type specific - in this case, it seems to be dependent on DEC205 expression. The fact that NF-κB exquisitely mediates fibroblastic complement C3 expression and acts as a repurposing of a signaling axis for chemotherapy resistance suggests the hardwiring of this pathway originates in mesenchymal cells.

The inventors describe the compositions, therapies, detection of mtDNA and gDNA and diagnostics of the present invention, based in part by these findings.

### Agents and Compositions

Various embodiments of the present invention provide for a protein. The protein is useful for binding cell free, circulating mtDNA, genomic DNA (gDNA) and depleting the circulating mtDNA and gDNA from circulation. The protein is structurally similar to an antibody wherein a fragment of the protein binds to circulating mtDNA, gDNA or both, and a fragment of the protein directs the entire protein to the liver for processing and removing of the mtDNA, gDNA or both. In various embodiments, these two fragments are on an antibody backbone to maintain or extend circulatory half-life.

Various embodiments of the present invention provide for a protein, comprising: a polypeptide that binds to mitochondrial DNA (mtDNA); and a Fc fragment of IgG receptor gamma (FcgRIIb) or a fragment thereof.

Various embodiments of the present invention provide for a protein, comprising: a polypeptide that binds to genomic DNA (gDNA); and a Fc fragment of IgG receptor gamma (FcgRIIb) or a fragment thereof.

Various embodiments of the present invention provide for a protein, comprising: a polypeptide that binds to both mitochondrial DNA (mtDNA) and genomic DNA (gDNA); and a Fc fragment of IgG receptor gamma (FcgRIIb) or a fragment thereof.

In various embodiments, the polypeptide that binds to mtDNA, gDNA or both comprises a fragment of DEC205 or a fragment of DEC205 with one or more amino acid deletions, additions or substitutions. In various embodiments, there are 1-10, 11-20, 21-30, 31-40, 41-50, 51-60, 61-70, 71-80, 81-90, or 91-100 amino acid deletions, additions or substitutions.

In various embodiments, the fragment of DEC205 is a polypeptide at least 90% identical to at least one domain selected from the group consisting of Ricin B-type lectin domain, fibronectin type II lectin domain, and at least one C-type lectin domain. In various embodiments, the fragment of DEC205 is a polypeptide at least 95, 96, 97, 98 or 99% identical to at least one domain selected from the group consisting of Ricin B-type lectin domain, fibronectin type II lectin domain, and at least one C-type lectin domain. In various embodiments, the fragment of DEC205 is a polypeptide comprising at least one domain selected from the group consisting of Ricin B-type lectin domain, fibronectin type II lectin domain, and at least one C-type lectin domain.

In various embodiments, the fragment of DEC205 is a polypeptide at least 90% identical to at least two domains selected from the group consisting of Ricin B-type lectin domain, fibronectin type II lectin domain, and at least one C-type lectin domain. In various embodiments, the fragment of DEC205 is a polypeptide at least 95, 96, 97, 98, or 99 % identical to at least two domains selected from the group consisting of Ricin B-type lectin domain, fibronectin type II lectin domain, and at least one C-type lectin domain. In various embodiments, the fragment of DEC205 is a polypeptide comprising at least two domains selected from the group consisting of Ricin B-type lectin domain, fibronectin type II lectin domain, and at least one C-type lectin domain.

In various embodiments, the fragment of DEC205 is a polypeptide at least 90% identical to at least three domains selected from the group consisting of Ricin B-type lectin domain, fibronectin type II lectin domain, and at least one C-type lectin domain. In various embodiments, the fragment of DEC205 is a polypeptide at least 95, 96, 97, 98 or 99% identical to at least three domains selected from the group consisting of Ricin B-type lectin domain, fibronectin type II lectin domain, and at least one C-type lectin domain. In various embodiments, the fragment of DEC205 is a polypeptide comprising at least three domains selected from the group consisting of Ricin B-type lectin domain, fibronectin type II lectin domain, and at least one C-type lectin domain.

In various embodiments, the fragment of DEC205 is a polypeptide at least 90% identical to Ricin B-type lectin domain, fibronectin type II lectin domain, or both. In various embodiments, the fragment of DEC205 is a polypeptide at least 95, 96, 97, 98 or 99% identical to Ricin B-type lectin domain, fibronectin type II lectin domain, or both. In various embodiments, the fragment of DEC205 is a polypeptide comprises Ricin B-type lectin domain, fibronectin type II lectin domain, or both.

In various embodiments, the fragment of DEC205 is a polypeptide at least 90% identical to Ricin B-type lectin domain and fibronectin type II lectin domain. In various embodiments, the fragment of DEC205 is a polypeptide at least 95, 96, 97, 98 or 99% identical to Ricin B-type lectin domain and fibronectin type II lectin domain. In various embodiments, the fragment of DEC205 is a polypeptide comprises Ricin B-type lectin domain and fibronectin type II lectin domain.

In various embodiments, the fragment of DEC205 is a polypeptide at least 90% identical to Ricin B-type lectin domain, fibronectin type II lectin domain, and at least one C-type lectin domain. In various embodiments, the fragment of DEC205 is a polypeptide at least 95, 96, 97, 98 or 99% identical to Ricin B-type lectin domain, fibronectin type II lectin domain, and at least one C-type lectin domain. In various embodiments, the fragment of DEC205 is a polypeptide comprising Ricin B-type lectin domain, fibronectin type II lectin domain, and at least one C-type lectin domain.

In various embodiments, the fragment of DEC205 is a polypeptide at least 90% identical to at least one C-type lectin domain. In various embodiments, the fragment of DEC205 is a polypeptide at least 95, 96, 97, 98 or 99% identical to at least one C-type lectin domain. In various embodiments, the fragment of DEC205 is a polypeptide comprising at least one C-type lectin domain.

In various embodiments, the fragment of DEC205 is a polypeptide at least 90% identical to at least two C-type lectin domains. In various embodiments, the fragment of DEC205 is a polypeptide at least 95, 96, 97, 98 or 99% identical to at least two C-type lectin domains. In various embodiments, the fragment of DEC205 is a polypeptide comprising at least two C-type lectin domains.

There are 10 C-type lectin domains on DEC205; thus, in various embodiments of the present invention, the at least one C-type lectin domain can be 1, 2, 3, 4, 5, 6, 7, 8, 9 or 10 C-type lectin domains.

In various embodiments, the fragment of DEC205 is a polypeptide at least 90, 95, 96, 97, 98 or 99% identical 3, 4, 5, 6, 7, 8, 9 or 10 C-type lectin domains. In various embodiments, the fragment of DEC205 is a polypeptide comprising 3, 4, 5, 6, 7, 8, 9 or 10 C-type lectin domains.

In various embodiments, the fragment of DEC205 comprises a polypeptide is at least 90% identical to a sequence selected from the group consisting of SEQ ID NO:1, SEQ ID NO:2, and SEQ ID NO:3. In various embodiments, the fragment of DEC205 comprises a polypeptide is at least 95, 96, 97, 98 or 99% identical to a sequence selected from the group consisting of SEQ ID NO:1, SEQ ID NO:2, and SEQ ID NO:3. In various embodiments, the fragment of DEC205 comprises a polypeptide that has a sequence selected from the group consisting of SEQ ID NO:1, SEQ ID NO:2, and SEQ ID NO:3.

In various embodiments, the fragment of DEC205 comprises a polypeptide is at least 90% identical to a sequence comprising SEQ ID NO:4. In various embodiments, the fragment of DEC205 comprises a polypeptide is at least 95, 96, 97, 98, or 99% identical to a sequence comprising SEQ ID NO:4. In various embodiments, the fragment of DEC205 comprises a polypeptide having the sequence as set forth in SEQ ID NO:4.

In various embodiments, the fragment of DEC205 comprises a polypeptide having at least 168 consecutive amino acids of SEQ ID NO:4. In various embodiments, the fragment of DEC205 comprises a polypeptide having 168 to 414 consecutive amino acids of SEQ ID NO:4. In various embodiments, the fragment of DEC205 comprises a polypeptide having 183 to 368 consecutive amino acids of SEQ ID NO:4. In various embodiments, the fragment of DEC205 comprises a polypeptide having 202 to 322 consecutive amino acids of SEQ ID NO:4. In various embodiments, the fragment of DEC205 comprises a polypeptide having 220 to 276 consecutive amino acids of SEQ ID NO:4. The determination of consecutive amino acids can start at amino acid number 1-292 of SEQ ID NO:4. For example, if it started at amino acid number 292, it will include all the amino acids until the end of SEQ ID NO:4. In various embodiments, these fragments of DEC205 has one or more amino acid additions, deletions or substitutions; for example, 1-5, 6-10, 11-15, 16-20 or 21-25 amino acid additions, deletions or substitutions.

In various embodiments, the Fc fragment of IgG receptor gamma (FcgRIIb) comprises a human IgG1 Fc domain, or a human IgG1 Fc domain with up to 22 amino acid additions, deletions, and/or substitutions. In various embodiments, it has 1, 2, 3, 4, 5, 6, 7, 8, 9, 10, 11, 12, 13, 14, 15, 16, 17, 18, 19, 20, 21, or 22 amino acid additions, deletions, and/or substitutions.

In various embodiments, the Fc fragment of IgG receptor gamma (FcgRIIb) or the fragment thereof comprises at least 205 consecutive amino acids as set forth in SEQ ID NO:5. In various embodiments, the Fc fragment of IgG receptor gamma (FcgRIIb) or the fragment thereof comprises 205-215, 216-227 consecutive amino acids as set forth in SEQ ID NO:5. The determination of consecutive amino acids can start at amino acid number 1-22.

In various embodiments, the Fc fragment of IgG receptor gamma (FcgRIIb) or the fragment thereof comprises a sequence with at least 90% sequence identity with SEQ ID NO:5. In various embodiments, the Fc fragment of IgG receptor gamma (FcgRIIb) or the fragment thereof comprises a sequence with at least 95, 96, 97, 98, or 99% sequence identity with SEQ ID NO:5. In various embodiments, the Fc fragment of IgG receptor gamma (FcgRIIb) or the fragment thereof comprises a polypeptide having the sequence as set forth in SEQ ID NO:5.

In various embodiments, the Fc fragment of IgG receptor gamma (FcgRIIb) comprises a polypeptide having the sequence as set forth in SEQ ID NO:5.

In various embodiments, the Fc fragment of IgG receptor gamma (FcgRIIb) is a mouse IgG1 Fc domain, or a mouse IgG1 Fc domain with up to 21 amino acid additions, deletions, and/or substitutions. In various embodiments, it has 1, 2, 3, 4, 5, 6, 7, 8, 9, 10, 11, 12, 13, 14, 15, 16, 17, 18, 19, 20, or 21 amino acid additions, deletions, and/or substitutions.

In various embodiments, the Fc fragment of IgG receptor gamma (FcgRIIb) or the fragment thereof comprises at least 209 consecutive amino acids as set forth in SEQ ID NO:6. In various embodiments, the Fc fragment of IgG receptor gamma (FcgRIIb) or the fragment thereof comprises 209-214, 215-219, 220-224, 225-229, 230-232 consecutive amino acids as set forth in SEQ ID NO:6. The determination of consecutive amino acids can start at amino acid number 1-23.

In various embodiments, the Fc fragment of IgG receptor gamma (FcgRIIb) or the fragment thereof comprises a sequence with at least 90% sequence identity with SEQ ID NO:6. In various embodiments, the Fc fragment of IgG receptor gamma (FcgRIIb) or the fragment thereof comprises a sequence with at least 95, 96, 07, 08, or 99% sequence identity with SEQ ID NO:6. In various embodiments, the Fc fragment of IgG receptor gamma (FcgRIIb) comprises a polypeptide having the sequence as set forth in SEQ ID NO:6.

In various embodiments, the protein further comprises a signal sequence, a linker, or both. In various embodiments, the signal sequence comprises the amino acids as set forth in SEQ ID NO:7. In various embodiments the signal sequence is at the N-terminus end of the protein. In various embodiment, the linker is between the polypeptide that binds to mitochondrial DNA (mtDNA), genomic DNA (gDNA), or both, and the Fc fragment of IgG receptor gamma (FcgRIIb) or the fragment thereof. In various embodiments, the linker is between the signal sequence and the polypeptide that binds to mitochondrial DNA (mtDNA), genomic DNA (gDNA), or both. In various embodiments the linker is 1, 2, 3, 4, 5, 6, 7, 8, 9, 10, 11, 12, 13, 14, or 15 amino acids in length.

In various embodiments, the protein is selected from a protein having the sequence as set forth in any one of SEQ ID NOs:8-13. In various embodiments, the protein is the protein having the sequence as set forth in SEQ ID NO:8. In various embodiments, the protein is a protein having a sequence at least 95, 96, 97, 98 or 99% identical to SEQ ID NO:8. In various embodiments, the protein is the protein having the sequence as set forth in SEQ ID NO:9. In various embodiments, the protein is a protein having a sequence at least 95, 96, 97, 98 or 99% identical to SEQ ID NO:9. In various embodiments, the protein is the protein having the sequence as set forth in SEQ ID NO:10. In various embodiments, the protein is a protein having a sequence at least 95, 96, 97, 98 or 99% identical to SEQ ID NO:10. In various embodiments, the protein is the protein having the sequence as set forth in SEQ ID NO:11. In various embodiments, the protein is a protein having a sequence at least 95, 96, 97, 98 or 99% identical to SEQ ID NO:11. In various embodiments, the protein is the protein having the sequence as set forth in SEQ ID NO: 12. In various embodiments, the protein is a protein having a sequence at least 95, 96, 97, 98 or 99% identical to SEQ ID NO:12. In various embodiments, the protein is the protein having the sequence as set forth SEQ ID NO:13. In various embodiments, the protein is a protein having a sequence at least 95, 96, 97, 98 or 99% identical to SEQ ID NO:13.

In various embodiments, the protein is the protein having the sequence as set forth in any one of amino acids 24-435 of SEQ ID NO:8, amino acids 24-583 of SEQ ID NO:9, amino acids 24-529 of SEQ ID NO:10, amino acids 24-440 of SEQ ID NO:11, amino acids 24-588 of SEQ ID NO:12, or amino acids 24-534 of SEQ ID NO: 13.

In various embodiments, the protein is selected from a protein comprising the sequence of SEQ ID NO:1 and SEQ ID NO:5; or SEQ ID NO:2 and SEQ ID NO:5; or SEQ ID NO:3 and SEQ ID NO:5; or SEQ ID NO:1 and SEQ ID NO:6; or SEQ ID NO:2 and SEQ ID NO:6; or SEQ ID NO:3 and SEQ ID NO:6.

In various embodiments, the polypeptide that binds to mtDNA, gDNA or both comprises a fragment of toll-like receptor 9 (TLR9) or a fragment of TLR9 with one or more amino acid deletions, additions or substitutions.

In various embodiments, the protein further comprises an Fc region of an antibody or a fragment thereof.

In various embodiments, the protein of the present invention is capable of depleting circulating mtDNA.

In various embodiments, the protein of the present invention is capable of depleting circulating genomic DNA (gDNA).

Various embodiments of the present invention provide for a nucleic acid encoding any one of the proteins of the present invention as described herein.

Various embodiments of the present invention provide for a cell for producing any one of the proteins of the present invention as described herein.

Various embodiments of the present invention provide for a cell comprising the nucleic acid encoding any one of the proteins of the present invention as described herein.

In various embodiments, the cell is a bacterial cell, a Chinese hamster ovarian cell (CHO) or a baby hamster kidney cell (BHK).

In various embodiments, the bacterial cell is Bacillus subtilis or Lactococus lactis. In various embodiments, the bacterial cell is a gram positive bacteria that make no endotoxin, which include but are not limited to: Lactococcus kimchii, other Lactococcus lactis subspecies; Lc. lactis subsp. cremoris, Lc. lactis subsp. hordniae, Lc. lactis subsp. lactis, and Lc. lactis subsp. tructae. Additional Bacillus include but are not limited to Bacillus clausii and Bacillus coagulans.

Various embodiments provide for a method of producing a protein of the present invention as described herein, comprising culturing a cell of the present invention as described herein; and isolating the protein from the cell or the cell culture media.

Various embodiments of the present invention provide for a combination, comprising: any one of the proteins of the present invention as described herein; and a therapeutic agent.

In various embodiments, the therapeutic agent is selected from the group consisting of an anti-tumor agent, a chemotherapeutic agent, an androgen ablating agent, a cardiac infarction treatment agent, a traumatic brain injury treatment agent, and combinations thereof. In various embodiments, the therapeutic agent is a taxane, anthracycline, or a platinum based antineoplastic drug. In various embodiments, the therapeutic agent is docetaxel, paclitaxel, cabazataxel, doxorubicin, epirubicin, idarubicin, valrubicin, cisplatin, oxaliplatin, carboplatin, irinotecan, or fluorouracil (5FU). In various embodiments, the therapeutic agent is an androgen receptor antagonist, an androgen synthesis inhibitor, or an anti-gonadotropin. In various embodiments, the therapeutic agent is selected from the group consisting of bicalutamide, enzalutamide, apalutamide, flutamide, nilutamide, darolutamide, cyproterone acetate, megestrol acetate, chlormadinone acetate, spironolactone, oxendolone, ketoconazole, abiraterone acetate, seviteronel, aminoglutethimide, finasteride, dutasteride, epristeride, alfatradiol, saw palmetto extract, leuprorelin, cetorelix and combinations thereof. In various embodiments, the therapeutic agent is aspirin, a thrombolytic agent, heparin, an antiplatelet agent, nitroglycerin, a beta blocker, an ACE inhibitor, a statin, and combinations thereof. In various embodiments, the therapeutic agent is a diuretic, an anti-seizure drug, a coma-inducing drug, or combinations thereof.

### Devices

Various embodiments of the present invention provide for a device, comprising: at least one inlet; at least one outlet; at least one chamber comprising a solid substrate; and any one of the proteins of the present invention as described herein, immobilized on the solid substrate.

In various embodiments, the device is a microfluidic device. In various embodiments, the solid substrate is dextran beads or sepharose beads.

Various embodiments of the present invention provide for a device, comprising any one of the proteins of the present invention as described herein, immobilized onto a solid substrate.

In various embodiments, the solid substrate is a multi-well plate. In various embodiments, the device is a plate suitable for an ELISA assay.

In various embodiments, the solid substrate is a bead. In various embodiments, the bead is suitable for a multiplex assay.

In various embodiments, the protein is further conjugated or immobilized to a conductive substrate to produce a detectable signal upon binding to mtDNA, gDNA, or both. In various embodiments, the conductive substrate is gold, silver, platinum, iridium, or copper. In various embodiments, the protein is further conjugated or immobilized to silicone.

In various embodiments, a device or system as described in International Application No. PCT/US2016/053145 filed Sep. 22, 2016, the entirety of which is herein incorporated by reference, is used to detect the mtDNA.

For example, a device comprising, consisting of or consisting essentially of a sample chamber having at least one analyte inlet, and a sensor component comprising an electrically conductive metal substrate or electrically conductive metal deposited or formed on a substrate. The conductive metal provides a reaction surface capable of binding circulating mtDNA having a functional group comprising sulfur or modified to comprise sulfur. The sensor component further comprises electrodes electrically coupled to the conductive metal and to a component for determining an electrical parameter of the metal, such as impedance, resistance, and/or conductance, subsequent to mtDNA binding to the metal surface. For example, if the parameter is impedance, the device further comprises a component for measuring impedance. The electrically conductive metal may be any suitable metal, but typically is selected from gold, silver, platinum, iridium, and combinations thereof, with gold being a particularly suitable metal. The electrically conductive metal may define a fluid flow path over which an analyte solution flows, the metal typically having a thickness of from 1 to 500 nanometers, a width of from 0.1 to about 20 millimeters, and a length of from about 0.1 to about 200 millimeters. The electrically conductive metal may be configured as a straight, curve, winding, and/or tortuous path. The sample chamber may define plural electrically insulated reaction surfaces. The device also may comprise plural sample chambers, arranged in parallel or in series. The disclosed embodiments can be a point of care device, and even more particularly a point of care device for detecting an amount of mtDNA in a sample from a subject.

Certain aspects of the present invention concern the recognition that a molecule reacting with a metal surface, such as a gold surface, induces an impedance change in the metal, and that impedance change can be directly correlated with the amount of the molecule reacting with the metal surface, or interacting with a capture molecule bound, typically covalently, to the metal surface. For example, the conductive metal substrate may comprise a receptor biomolecule coupled to a portion of the metal surface through a thiol functional group. In such embodiments, a remaining portion of the metal surface may comprise a blocking agent, such as a thiolated polyethylene glycol, to preclude target molecule binding to the surface. In certain embodiments, the receptor molecule is a peptide, such as an antibody or extracellular receptor domain, that is coupled to the metal surface. One method of coupling a peptide to the surface is by modifying the peptide to include at least one pendant cysteine.

Systems comprising embodiments of the disclosed device also are disclosed. Disclosed systems may include a sensor device that defines a disposable sensor unit comprising the electrically conductive metal for coupling to a detection device for detecting a change in an electrical parameter of the conductive metal subsequent to mtDNA binding. Alternatively, the system can comprise a reusable sensor unit comprising the electrically conductive metal. Disclosed systems can further comprise one or more of a central processing unit for controlling functions of the system; a temperature sensor; a data storage unit; a fluid pump for flowing analyte and/or enzyme solutions to and/or through the device; a sample collector; a sample reservoir or cartridge; one or more filtration modules positioned to filter a fluid stream into the system or between components of the system; an enzyme reservoir or cartridge; an enzyme reaction module; a buffer reservoir or cartridge; a power supply; and combinations thereof.

Certain disclosed method embodiments comprise using the device or system to measure an mtDNA in a sample. The mtDNA typically comprises a functional group comprising a sulfur atom or modified to comprise a sulfur atom. Alternatively, the mtDNA may have a functional group that is converted to a thiol enzymatically, chemically or thermally. As yet another alternative, the mtDNA may be reacted with cysteine to provide a terminal cysteine moiety for detection and measurement using the device.

The mtDNA is detected, and the mtDNA amount quantified, using an electrical parameter. If the electrical parameter is impedance, the measured impedance value may be correlated with an mtDNA amount in the sample, such as by using a standard curve.

Certain disclosed embodiments comprise using a device wherein the conductive metal substrate comprises a receptor biomolecule coupled to a portion of the metal surface through a thiol functional group. A remaining portion of the metal surface may comprise a blocking agent to preclude target molecule binding to the surface. The receptor molecule may be, for example, a peptide or an extracellular receptor domain that is coupled to the metal surface by cysteine. The peptide may be modified to include a pendant cysteine amino acid.

### Methods

Various embodiments of the present invention provide for methods of treatment. Various methods combine a therapeutic agent with a circulating mtDNA depleting agent to treat a patient. As discussed, mtDNA is expelled by cells undergoing stress caused by a therapeutic agent that is used to treat the disease or condition. As such, an increase in circulating mtDNA which promotes and inflammatory cascade which impacts tumor expansion and therapeutic resistance. While not wishing to be bound by any particular theory, depleting mtDNA from circulation allows for the therapeutic agent to continue working and/or allows for decreases tumor expansion.

Various embodiments of the present invention provide for a method of treating a disease or condition, comprising: administering a protein of the present invention to a mammalian subject to treat the disease or condition.

Various embodiments of the present invention provide for a method of treating a disease or condition, comprising: administering a combination of a protein of the present invention and a therapeutic agent to a mammalian subject to treat the disease or condition.

In various embodiments, the disease or condition is selected from the group consisting of a tumor, cancer, cardiac infarct, and traumatic brain injury.

In various embodiments, the cancer is a solid tumor cancer. In various embodiments, the cancer is prostate cancer or breast cancer.

Various embodiments of the present invention provide for a method of reducing circulating mitochondrial DNA (mtDNA) in a mammalian subject, comprising: administering any one of the proteins of the present invention as described herein to the mammalian subject.

Various embodiments of the present invention provide for a method of reducing circulating mitochondrial DNA (mtDNA) in a mammalian subject, comprising: administering any one of the combination of the present invention as described herein to the mammalian subject.

Various embodiments of the present invention provide for a method of reducing circulating mitochondrial DNA (mtDNA) in a mammalian subject, comprising: removing circulating mtDNA from the mammalian subject’s blood.

Various embodiments of the present invention provide for a method of reducing circulating mitochondrial DNA (mtDNA) in a mammalian subject, comprising: administering any one of the bacterial cells of the present invention as described herein.

Various embodiments of the present invention provide for a method of reducing circulating genomic DNA (gDNA) in a mammalian subject, comprising: administering any one of the proteins of the present invention as described herein to the mammalian subject.

Various embodiments of the present invention provide for a method of reducing circulating genomic DNA (gDNA) in a mammalian subject, comprising: administering any one of the combination of the present invention as described herein to the mammalian subject.

Various embodiments of the present invention provide for a method of reducing circulating genomic DNA (gDNA) in a mammalian subject, comprising: removing circulating mtDNA from the mammalian subject’s blood.

Various embodiments of the present invention provide for a method of reducing circulating genomic DNA (gDNA) in a mammalian subject, comprising: administering any one of the bacterial cells of the present invention as described herein.

In various embodiments, the mammalian subject has or is suspected to have a disease or condition caused by or related to elevated levels of circulating mitochondrial DNA (mtDNA). In various embodiments, the mammalian subject has or is suspected to have a disease or condition caused by or related to elevated levels of genomic DNA (gDNA).

In various embodiments, the mammalian subject has or is suspected to have a disease or condition caused by or related to elevated levels of circulating mitochondrial DNA (mtDNA) and genomic DNA (gDNA).

In various embodiments, the disease or condition caused by or related to elevated levels of mtDNA, gDNA, or both, is selected from the group consisting of a tumor, cancer, cardiac infarct, cardiac disease, physical trauma, traumatic brain injury, infection, stroke, inflammation, autoimmune disease, cachexia, and lupus. In various embodiments, the disease or condition caused by or related to elevated levels of mtDNA is selected from the group consisting of a tumor, cancer, cardiac infarct, cardiac disease, physical trauma, traumatic brain injury, infection, stroke, inflammation, autoimmune disease, and cachexia. In various embodiments, the disease or condition caused by or related to elevated levels of gDNA is lupus.

In various embodiments, the cancer is a solid tumor cancer. In various embodiments, the cancer is prostate cancer or breast cancer.

In various embodiments, removing circulating mtDNA from the mammalian subject’s blood comprises passing the subject’s blood through any one of the devices of the present invention.

In various embodiments, removing circulating mtDNA may be done in conjunction with chemotherapy, which can sensitize the subject to the chemotherapy. For example, one or more cycles of treatment to remove mtDNA may be given to the subject. In a non-limiting example, a first cycle can be on days 1 and 4 having initial doses of 3 mg/kg IV for one dose on day 1, followed by 7 mg/kg on day 4, followed by full dose regimen of 10 mg/kg IV for one dose on days 8, 15 and 22. The second cycle can be 10 mg/kg IV for one dose on days 1, 8, 15 and 22. These calculations for dosages are based on a max weight of 85 kg. One of skill in the art can adjust dosages based on the subject’s weight and health. As such, in various embodiments, the method comprises removing circulating mtDNA from the subject’s blood, and administering a chemotherapeutic treatment to the subject.

Various embodiments of the present invention provide for a method of measuring circulating mitochondrial DNA (mtDNA), genomic DNA, or both comprising: obtaining a biological sample; contacting any one of the proteins of the present invention as described herein to the biological sample; detecting the binding of the protein to the mtDNA, gDNA, or both; and quantifying the amount of protein-mtDNA binding conjugate, protein-gDNA binding conjugate, or both.

In various embodiments, the protein further comprises a label to produce a detectable signal. The label can be any label as exemplified herein.

In various embodiments, the detectable signal is colorimetric, fluorescence, or luminescence.

In various embodiments, the protein is contacted to the biological sample using any one of the devices of the present invention as described herein.

In various embodiments, the device comprises a conductive substrate and the protein is conjugated or immobilized to the conductive substrate to produce a detectable signal upon binding to mtDNA, gDNA or both, wherein the detectable signal is impedance, resistance, change in current, or change in electrochemical impedance spectrum, and wherein the conductive substrate is selected from the group consisting of gold, silver, platinum, iridium, copper.

In various embodiments, the method of measuring circulating mitochondrial DNA (mtDNA), genomic DNA, or both comprises using an ELISA based assay. In various embodiments, the method of measuring circulating mitochondrial DNA (mtDNA), genomic DNA, or both comprises using a multiplex based assay.

Various embodiments also provide for a method of measuring circulating mtDNA. These methods can be useful to identify subject who are in need of an mtDNA depleting agent of the present invention.

Various embodiments provide for a method of measuring circulating mitochondrial DNA (mtDNA), comprising: obtaining a biological sample; contacting a protein of the present invention to the biological sample; detecting the binding of the protein to the mtDNA; and quantifying the amount of protein-mtDNA.

In various embodiments, the protein further comprises a label to produce a detectable signal. A label can be any label as exemplified herein.

In various embodiments, the protein is further conjugated to a conductive substrate to produce a detectable signal upon binding to mtDNA. In various embodiments, the conductive substrate is gold, silver, platinum, iridium, or copper. In various embodiments the protein is further conjugated to silicone. In various embodiments, the detectable signal is impedance, resistance, conductance, change in current, or change in electrochemical impedance spectrum.

In various embodiments, the present invention provides pharmaceutical compositions including a pharmaceutically acceptable excipient along with a therapeutically effective amount of the inventive protein of the present invention, or the combination of the present invention. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions comprise one or more of surfactants (e.g., polysorbate 20 and 80), carbohydrates (e.g., cyclodextrin derivatives) and amino acids (e.g., arginine and histidine) can help prevent aggregation by this mechanism. Other components can be used to stabilize the protein including but not limited to: cyclodextrin, pluronic F68, trehalose, glycine and amino acids such as arginine, glycine, glutamate and histidine.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals such as sodium, lithium, potassium, calcium, magnesium and the like, as well as nontoxic ammonium, quaternary ammonium, and amine cations including, but not limited to ammonium, tetramethylanunonium, tetraethyl ammonium, methyl amine, dimethyl amine, trimethylamine, triethylamine, ethylamine, and the like (see, e.g., Berge S. M., et al. (1977) J. Pharm. Sci. 66, 1, which is incorporated herein by reference).

The term “pharmaceutically acceptable esters” refers to the relatively nontoxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

As used herein, “pharmaceutically acceptable salts or prodrugs” are salts or prodrugs that are, within the scope of sound medical judgment, suitable for use in contact with the tissues of subject without undue toxicity, irritation, allergic response, and the like, commensurate with a reasonable benefit/risk ratio, and effective for their intended use.

The term “prodrug” refers to compounds that are rapidly transformed in vivo to yield the functionally active one or more peptides as disclosed herein or a mutant, variant, analog or derivative thereof. A thorough discussion is provided in T. Higachi and V. Stella, “Pro-drugs as Novel Delivery Systems,” Vol. 14 of the A. C. S. Symposium Series, and in Bioreversible Carriers in: Drug Design, ed. Edward B. Roche, American Pharmaceutical Association and Pergamon Press, 1987, both of which are hereby incorporated by reference. As used herein, a prodrug is a compound that, upon in vivo administration, is metabolized or otherwise converted to the biologically, pharmaceutically or therapeutically active form of the compound. A prodrug of the one or more peptides as disclosed herein or a mutant, variant, analog or derivative thereof can be designed to alter the metabolic stability or the transport characteristics of one or more peptides as disclosed herein or a mutant, variant, analog or derivative thereof, to mask side effects or toxicity, to improve the flavor of a compound or to alter other characteristics or properties of a compound. By virtue of knowledge of pharmacodynamic processes and drug metabolism in vivo, once a pharmaceutically active form of the one or more peptides as disclosed herein or a mutant, variant, analog or derivative thereof, those of skill in the pharmaceutical art generally can design prodrugs of the compound (see, e.g., Nogrady (1985) Medicinal Chemistry A Biochemical Approach, Oxford University Press, N. Y., pages 388-392). Conventional procedures for the selection and preparation of suitable prodrugs are described, for example, in “Design of Prodrugs,” ed. H. Bundgaard, Elsevier, 1985. Suitable examples of prodrugs include methyl, ethyl and glycerol esters of the corresponding acid.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or nonaqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject’s response to administration of a compound and adjusting the dosage accordingly. For additional guidance, see Remington: The Science and Practice of Pharmacy (Gennaro ed. 20th edition, Williams & Wilkins PA, USA) (2000).

### Kits

The present invention is also directed to a kit to treat a disease or condition as described herein or for measuring the amount of circulating mtDNA, gDNA, or both. The kit is useful for practicing the inventive method of treating a disease or condition as described herein, or for measuring the amount of circulating mtDNA, gDNA, or both. The kit is an assemblage of materials or components, including at least one of the inventive compositions. Thus, in some embodiments the kit contains a composition including the inventive protein, as described above.

The exact nature of the components configured in the inventive kit depends on its intended purpose. For example, some embodiments are configured for the purpose of treating the disease or condition, and some embodiments are configured for the purposes of measuring circulating mtDNA, gDNA, or both. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to provide a desired outcome, such as to treat a disease or condition, or to measure circulating mtDNA, gDNA, or both. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing the inventive protein of the present invention, or the combination of the present invention. The packaging material generally has an external label which indicates the contents and/or purpose of the kit and/or its components.

Table of Sequences

| Name | Sequence | SEQ ID NO: |
|---|---|---|
| RF | MSGRAANDPFTIVHGNTGKCIKPVYGWIVADDCDETEDKLWKWVS QHRLFHLHSQKCLGLDITKSVNELRMFSCDSSAMLWWKCEHHSLYG AARYRLALKDGHGTAISNASDVWKKGGSEESLCDQPYHEIYTRDGN SYGRPCEFPFLIDGTWHHDCILDEDHSGPWCATTLNYEYDRKWGIC | 1 |
| RFL | MSGRAANDPFTIVHGNTGKCIKPVYGWIVADDCDETEDKLWKWVS QHRLFHLHSQKCLGLDITKSVNELRMFSCDSSAMLWWKCEHHSLYG AARYRLALKDGHGTAISNASDVWKKGGSEESLCDQPYHEIYTRDGN SYGRPCEFPFLIDGTWHHDCILDEDHSGPWCATTLNYEYDRKWGICL KPENGCEDNWEKNEQFGSCYQFNTQTALSWKEAYVSCQNQGADLL SINSAAELTYLKEKEGIAKIFWIGLNQLYSARGWEWSDHKPLNFLNW DPDRPSAPTIGGSSCARMDAESGLWQSFSCEAQLPYVCRKPLNNTVY PYDVPDYA | 2 |
| 2L | MELTDVWTYSDTRCDAGWLPNNGFCYLLVNESNSWDKAHAKCKA FSSDLISIHSLADVEVVVTKLHNEDIKEEVWIGLKNINIPTLFQWSDGT EVTLTYWDENEPNVPYNKTPNCVSYLGELGQWKVQSCEEKLKYVC KRKGEKLNDASSDKMCPPDEGWKRHGETCYKIYEDEVPFGTNCNLT ITSRFEQEYLNDLMKKYDKSLRKYFWTGLRDVDSCGEYNWATVGG RRRAVTFSNWNFLEPASPGGCVAMSTGKSVGKWEVKDCRSFKALSI CK | 3 |
| RF2L | MSGRAANDPFTIVHGNTGKCIKPVYGWIVADDCDETEDKLWKWVS QHRLFHLHSQKCLGLDITKSVNELRMFSCDSSAMLWWKCEHHSLYG AARYRLALKDGHGTAISNASDVWKKGGSEESLCDQPYHEIYTRDGN SYGRPCEFPFLIDGTWHHDCILDEDHSGPWCATTLNYEYDRKWGIC MELTDVWTYSDTRCDAGWLPNNGFCYLLVNESNSWDKAHAKCKA FSSDLISIHSLADVEVVVTKLHNEDIKEEVWIGLKNINIPTLFQWSDGT EVTLTYWDENEPNVPYNKTPNCVSYLGELGQWKVQSCEEKLKYVC KRKGEKLNDASSDKMCPPDEGWKRHGETCYKIYEDEVPFGTNCNLT ITSRFEQEYLNDLMKKYDKSLRKYFWTGLRDVDSCGEYNWATVGG RRRAVTFSNWNFLEPASPGGCVAMSTGKSVGKWEVKDCRSFKALSI CK | 4 |
| human IgG1 Fc domain (Chain A, Ig Gamma-1 Chain C region) | DKTHTCPPCPAPELLGGPSVFLFPPKPKDTLMISRTPEVTCVVVDVSH EDPEVKFNWYVDGVEVHNAKTKPREEQYNSTYRVVSVLTVLHQDW LNGKEYKCKVSNKALPAPIEKTISKAKGQPREPQVYTLPPSREEMTK NQVSLTCMVEGFYPSDIAVEWESNGQPENNYKTTPPVLDSDGSFFLY SKLTVDKSRWQQGNVFSCSVMHEALHNHYTQKSLSLSPGK | 5 |
| mouse IgG1 Fc domain | PRGPTIKPCPPCKCPAPNLLGGPSVFIFPPKIKDVLMISLSPIVTCVVVD VSEDDPDVQISWFVNNVEVHTAQTQTHREDYNSTLRVVSALPIQHQ DWMSGKEFKCKVNNKDLPAPIERTISKPKGSVRAPQVYVLPPPEEEM TKKQVTLTCMVTDFMPEDIYVEWTNNGKTELNYKNTEPVLDSDGSY FMYSKLRVEKKNWVERNSYSCSVVHEGLHNHHTTKSFSRTPGK | 6 |
| Signal sequence | MYRMQLLSCIALSLALVTNS | 7 |
| DEC205-RF-Fc, human | MYRMQLLSCIALSLALVTNSAIAMSGRAANDPFTIVHGNTGKCIKPV YGWIVADDCDETEDKLWKWVSQHRLFHLHSQKCLGLDITKSVNELR MFSCDSSAMLWWKCEHHSLYGAARYRLALKDGHGTAISNASDVWK KGGSEESLCDQPYHEIYTRDGNSYGRPCEFPFLIDGTWHHDCILDEDH SGPWCATTLNYEYDRKWGICLEDKTHTCPPCPAPELLGGPSVFLFPP KPKDTLMISRTPEVTCVWDVSHEDPEVKFNWYVDGVEVHNAKTKP REEQYNSTYRVVSVLTVLHQDWLNGKEYKCKVSNKALPAPIEKTISK AKGQPREPQVYTLPPSREEMTKNQVSLTCMVEGFYPSDIAVEWESNG QPENNYKTTPPVLDSDGSFFLYSKLTVDKSRWQQGNVFSCSVMHEA LHNHYTQKSLSLSPGK | 8 |
| DEC205 RFL-Fc, human | MYRMQLLSCIALSLALVTNSAIAMSGRAANDPFTIVHGNTGKCIKPV YGWIVADDCDETEDKLWKWVSQHRLFHLHSQKCLGLDITKSVNELR MFSCDSSAMLWWKCEHHSLYGAARYRLALKDGHGTAISNASDVWK KGGSEESLCDQPYHEIYTRDGNSYGRPCEFPFLIDGTWHHDCILDEDH SGPWCATTLNYEYDRKWGICLKPENGCEDNWEKNEQFGSCYQFNT QTALSWKEAYVSCQNQGADLLSINSAAELTYLKEKEGIAKIFWIGLN QLYSARGWEWSDHKPLNFLNWDPDRPSAPTIGGSSCARMDAESGLW QSFSCEAQLPYVCRKPLNNTVYPYDVPDYALEDKTHTCPPCPAPELL GGPSVFLFPPKPKDTLMISRTPEVTCVVVDVSHEDPEVKFNWYVDGV EVHNAKTKPREEQYNSTYRVVSVLTVLHQDWLNGKEYKCKVSNKA LPAPIEKTISKAKGQPREPQVYTLPPSREEMTKNQVSLTCMVEGFYPS DIAVEWESNGQPENNYKTTPPVLDSDGSFFLYSKLTVDKSRWQQGN VFSCSVMHEALHNHYTQKSLSLSPGK | 9 |
| DEC205-2L-Fc, human | MYRMQLLSCIALSLALVTNSAIAMELTDVWTYSDTRCDAGWLPNNG FCYLLVNESNSWDKAHAKCKAFSSDLISIHSLADVEVVVTKLHNEDI KEEVWIGLKNINIPTLFQWSDGTEVTLTYWDENEPNVPYNKTPNCVS YLGELGQWKVQSCEEKLKYVCKRKGEKLNDASSDKMCPPDEGWKR HGETCYKIYEDEVPFGTNCNLTITSRFEQEYLNDLMKKYDKSLRKYF WTGLRDVDSCGEYNWATVGGRRRAVTFSNWNFLEPASPGGCVAMS TGKSVGKWEVKDCRSFKALSICKLEDKTHTCPPCPAPELLGGPSVFLF PPKPKDTLMISRTPEVTCVVVDVSHEDPEVKFNWYVDGVEVHNAKT KPREEQYNSTYRVVSVLTVLHQDWLNGKEYKCKVSNKALPAPIEKTI SKAKGQPREPQVYTLPPSREEMTKNQVSLTCMVEGFYPSDIAVEWES NGQPENNYKTTPPVLDSDGSFFLYSKLTVDKSRWQQGNVFSCSVMH EALHNHYTQKSLSLSPGK | 10 |
| DEC205-RF-Fc, mouse | MYRMQLLSCIALSLALVTNSAIAMSGRAANDPFTIVHGNTGKCIKPV YGWIVADDCDETEDKLWKWVSQHRLFHLHSQKCLGLDITKSVNELR MFSCDSSAMLWWKCEHHSLYGAARYRLALKDGHGTAISNASDVWK KGGSEESLCDQPYHEIYTRDGNSYGRPCEFPFLIDGTWHHDCILDEDH SGPWCATTLNYEYDRKWGICLEPRGPTIKPCPPCKCPAPNLLGGPSVF IFPPKIKDVLMISLSPIVTCVVVDVSEDDPDVQISWFVNNVEVHTAQT QTHREDYNSTLRVVSALPIQHQDWMSGKEFKCKVNNKDLPAPIERTI SKPKGSVRAPQVYVLPPPEEEMTKKQVTLTCMVTDFMPEDIYVEWT NNGKTELNYKNTEPVLDSDGSYFMYSKLRVEKKNWVERNSYSCSV VHEGLHNHHTTKSFSRTPGK | 11 |
| DEC205 RFL-Fc, mouse | MYRMQLLSCIALSLALVTNSAIAMSGRAANDPFTIVHGNTGKCIKPV YGWIVADDCDETEDKLWKWVSQHRLFHLHSQKCLGLDITKSVNELR MFSCDSSAMLWWKCEHHSLYGAARYRLALKDGHGTAISNASDVWK KGGSEESLCDQPYHEIYTRDGNSYGRPCEFPFLIDGTWHHDCILDEDH SGPWCATTLNYEYDRKWGICLKPENGCEDNWEKNEQFGSCYQFNT QTALSWKEAYVSCQNQGADLLSINSAAELTYLKEKEGIAKIFWIGLN QLYSARGWEWSDHKPLNFLNWDPDRPSAPTIGGSSCARMDAESGLW QSFSCEAQLPYVCRKPLNNTVYPYDVPDYALEPRGPTIKPCPPCKCPA PNLLGGPSVFIFPPKIKDVLMISLSPIVTCVVVDVSEDDPDVQISWFVN NVEVHTAQTQTHREDYNSTLRVVSALPIQHQDWMSGKEFKCKVNN KDLPAPIERTISKPKGSVRAPQVYVLPPPEEEMTKKQVTLTCMVTDF MPEDIYVEWTNNGKTELNYKNTEPVLDSDGSYFMYSKLRVEKKNW VERNSYSCSVVHEGLHNHHTTKSFSRTPGK | 12 |
| DEC205-2L-Fc, mouse | MYRMQLLSCIALSLALVTNSAIAMELTDVWTYSDTRCDAGWLPNNG FCYLLVNESNSWDKAHAKCKAFSSDLISIHSLADVEVVVTKLHNEDI KEEVWIGLKNINIPTLFQWSDGTEVTLTYWDENEPNVPYNKTPNCVS YLGELGQWKVQSCEEKLKYVCKRKGEKLNDASSDKMCPPDEGWKR HGETCYKIYEDEVPFGTNCNLTITSRFEQEYLNDLMKKYDKSLRKYF WTGLRDVDSCGEYNWATVGGRRRAVTFSNWNFLEPASPGGCVAMS TGKSVGKWEVKDCRSFKALSICKLEPRGPTIKPCPPCKCPAPNLLGGP SVFIFPPKIKDVLMISLSPIVTCVVVDVSEDDPDVQISWFVNNVEVHT AQTQTHREDYNSTLRVVSALPIQHQDWMSGKEFKCKVNNKDLPAPI ERTISKPKGSVRAPQVYVLPPPEEEMTKKQVTLTCMVTDFMPEDIYV EWTNNGKTELNYKNTEPVLDSDGSYFMYSKLRVEKKNWVERNSYS CSVVHEGLHNHHTTKSFSRTPGK | 13 |

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Animal experiments and cultured cells: Male C57BL/6 mice aged 7-8 weeks were housed in a pathogen-free environment at the Cedars-Sinai Medical Center Animal Facility under the approval of the Institutional Animal Care and Use Committee (# 3679). Sub-renal capsule was performed with wild type mouse fibroblasts (6×10⁵) or TLR9^{-/-} mouse fibroblasts (6×10⁵) combining with mouse prostate epithelial cell TRAMP-C2 (2×10⁵) . Treatment with SB290157 (1 mg/kg; i.p. daily) was started after two weeks of grafting and continued for five weeks. All mouse kidney, spleen, lymph nodes were harvested after five weeks of treatment were fixed in paraffin embedded for IHC or dissociated for FACS analysis. Subcutaneous xenograft was done in male nude mice aged 7-8 weeks in combination of PC3 (5×10⁵) and CAF (15×10⁵). Grafts were monitored by caliper throughout the time course of treatment with docetaxel (6 mg/kg/week) and SB290157 (1 mg/kg; IP, every day). Harvested tissues were fixed in paraffin embedded for IHC or dissociated for immunoblot analysis.

Cultured primary NAF and CAF (derived in our laboratory) were treated with LNCaP-CM, CpG-ODN (5 µM, InvivoGen, San Diego, CA), docetaxel (10 nM, SanofiAventis), N-acetylcysteine (10 mM, Sigma-Aldrich, St. Louis, MO), SB290157 (1 µM, Calbiochem) for 48 hours. Conditioned medium was treated with DNase1 (0.1 mg/ml, Sigma-Aldrich) in 37° C. for 1 hour followed by heat inactivation.

Immunodetection: Paraffin embedded tissue were processed for Immunohistochemical localization was performed with antibodies against p-AKT, p-TAK, p-histoneH3 (Cell Signaling, Danvers, MA), C3 (Santa Cruz Biotechnology, Santa Cruz, CA), and TUNEL (Thermo Fisher Scientific Inc.) as previously described before (52, 53). All the slides were scanned using Leica SCN400 (Leica Micro System, Buffalo Grove, IL) and analyzed by Tissue IA Optimizer (Leica). The values of positively stained cells were measured in an unbiased manner. C3a concentration of cultured medium and serum was assayed by sandwich ELISA using human C3a ELISA kit (BD Bioscience, San Jose, CA) according to the manufacturer’s instructions. Western blots separated by 10, 12, or 15% SDS-polyacrylamidegels were incubated with primary antibodies for TLR9, DEC205 (LS Bio Seattle, WA), phospho-TAK1, TAK1, phospho-AKT, AKT, phosphor-ERK1/2, ERK, BCL2, beclin, CHOP (Cell Signaling), LC3 (Abcam, Cambridge, MA), C3 (Santa Cruz Biotechnology) and p62 (ProgenBiotechnik, Heidelberg, Germany). Western blots were visualized using alkaline phosphatase-conjugated secondary antibodies (Sigma-Aldrich). The ELISA for anaphylatoxin C3a for performed according to manufacturer guidelines (LSBio Inc.).

DNA quantitation: Total DNA from serum or cultured medium was isolated by quick-cfDNA™ serum and plasma kit (Zymo Research, Irvine, CA). The purified total DNA from serum and cultured medium were PCR amplified by mitochondria specific MT-CO2 gene (using the following primers: 5'- CCT GCG ACT CCT TGA CGT TG-3' (SEQ ID NO:14) and 5'- AGC GGT GAA AGT GGT TTG GTT-3' (SEQ ID NO:15)). Quantitation was achieved through the use of a standard curve method by realtime PCR. Telomere-specific sequence (TTAGGG)₁₄ (SEQ ID NO:16) was measured using the TRAPEZE® RT Telomerase Detection Kit (Millipore, Burlington, MA).

Mitochondrial DNA immune precipitation (mDIP): We followed the manufacturer’s ChIP protocol of Zymo-Spin CHIP Kit (Zymo Research). Briefly, mtDNA from conditioned medium was immunoprecipitated either by normal rabbit IgG antibodies as a negative control or anti DEC205 antibody (Santa Cruz Biotechnology). 100 ng of mitochondrial DNA was added to condition medium as a positive control. Non-immunoprecipitated DNA was used as total input control. The purified immunoprecipitated DNA was PCR amplified by mitochondria specific primers (MT-CO2) as mentioned above and was compared to input DNA.

Detection of reactive oxygen species: FACS and fluorescent staining was performed for ROS detection in CAF by using 2',7'-Dichlorofluorescin Diacetate (H2-DCFDA) (Sigma-Aldrich). Cells were labeled with 10 µM H2-DCFDA for 30 minutes at 37° C. in dark and ROS production was monitored under fluorescence microscopy and quantified through flow cytometric analysis. FlowJo software (Tree Star Inc. Ashland, OR) was used for FACS analysis.

Catalase activity assay: Catalase activity was measured in CAF lysate by using OxiSelect™ Catalase Activity Assay Kit (Cell Biolabs, INC San Diego, CA) according to manufacture protocol and absorbance was taken 520 nm in a 96 well pate. 3-Amino-1,2,4-triazole (Santa Cruz Biotechnology) 10 mM was used as catalase inhibitor.

3D organotypic co-culture: 3D organotypic co-culture was performed in a collagen matrix. PC3 and CAF were combined in a 1:3 ratio in collagen matrix contain 50% rat tail collagen I, 20% of matrigel, 10% of 10x DMEM medium and 1x ready DMEM 5%, 1x ready RPMI 5%, FBS 5% and Nu serum 5%. Cells were treated with docetaxel and SB290157 for 48 hours after 72 hours of expansion in the matrix. The cells were dissociated from the matrix with collagenase and dispase for Ki67 FACS analysis.

Statistical analysis: Experiments were done a minimum of three times. Results were shown in terms of mean ± S.D. Student’s t-test and one-way ANOVA were used for comparisons among groups and repeated measured ANOVA was applied for determining the significance of two or more data series. Statistical tests utilized are reported in the figure legends, along with the associated P values was performed by using Origin software (OriginLab, Northampton, MA). Cell viability was examined by using MTT assay as indicated by the manufacturer (Thermo Fisher, Canoga Park, CA) for the calculation of synergistic drug interactions was performed by Chou-Talalay method (R Package).

### Example 2

Activation of TLR9 and anaphylatoxin C3a in cancer associated fibroblast through mitochondrial DNA. Based on reported elevation of mtDNA in PCa patient blood, we measured the mtDNA content in the conditioned media of prostate cell lines. We found that PCa lines (PC3, LNCaP, and TRAMPC2) expressed 3 to 10 times more mtDNA in the conditioned media than a benign prostate epithelial cell line, BPH1 (FIG. 1A). To determine if there was a paracrine mechanism for PCa epithelial proliferation, we incubated CAF with conditioned media from PCa epithelia. We tested for the expression of the mtDNA cognate receptor, TLR9 and its downstream effectors. TLR9 mRNA expression by CAF was only found to be significantly upregulated by LNCaP-conditioned media (CM), compared to normal prostate tissue associated fibroblasts (NAF) or treatment of either CAF/NAF with BPH1-CM (FIG. 7A). Examining the DNA content of the LNCaP-CM, we found mtDNA to be approximately 10-fold greater than telomeric DNA (FIG. 7B). PCa epithelial conditioned media treatment of CAF resulted in upregulation of TLR9 and downstream phosphorylated-TAK1, NF-κB p65 phosphorylation, cleaved-caspase1 and IL-1β protein expression (FIG. 1B and FIG. 7C). Sonication of the conditioned media did not significantly alter the TLR9 expression when compared to DNase treatment indicated that exosome-based signaling may not be involved (FIG. 7D). This was further verified by inhibiting exosome generation by the LNCaP cells with dynasore (dynamin inhibitor) resulting in no appreciable changes the mtDNA content in the media (FIG. 7E). Heat inactivation alone was used as a control as it can activate growth factors in serum. Since TLR9 is a cytoplasmic receptor, we sought to identify a mediator for DNA entry into the cell. Candidate mediators with the capacity to bind DNA, such as HMGB1, HMGA2, and DEC205 were found to be expressed by CAF in response to LNCaP-CM (FIG. 7F). HMGB1 expression was similarly induced by both NAF and CAF cells in response to LNCaP-CM, but HMGA2 expression was constitutively expressed regardless of LNCaP-CM treatment. LNCaP-CM effectively induced the DEC205 in CAF, but not NAF (FIG. 1C). DEC205 is a transmembrane endocytic receptor reported to bind and internalize unmethylated-CpG by dendritic cells. We tested whether mtDNA could bind DEC205 in CAF cells by adapting the methodology of a chromatin immunoprecipitation assay, we termed mtDNA immune precipitation (mDIP). We were able to PCR amplify the mitochondrial MT-CO2 gene following immunoprecipitation of DEC205 in the presence of LNCaP-CM, but not in its absence (FIG. 1D). Following the discovery that NF-κB signaling in CAF is a result of PCa-derived mtDNA, we performed a focused qPCR array to determine the effect of NF-κB on downstream target genes. As expected, LNCaP-CM induced the expression of multiple inflammatory cytokines by CAF, including IL-6, CXCL8, and CCL11 (FIG. 1E). Interestingly, complement C3 was the highest differentially expressed CAF gene by >12 Log-fold, depicted by the volcano plot (FIG. 1F). The role of complement C3 in fighting invasive pathogens is well described. More recently, C3 has been implicated in potentiating tumor cell growth. However, the active component, anaphylatoxin C3a, is a product of a tightly regulated proteolytic cleavage of C3. Interestingly, we found that LNCaP-CM induced TLR9 and C3a expression was sensitive to DNase treatment (FIG. 1G). Thus, mtDNA secreted by PCa epithelia can bind DEC205 on the cell surface of CAF and is associated with TLR9 and C3a maturation (FIG. 1H).

Critically, C3a is reported to promote cancer epithelial proliferation, but the pathway of tumor-associated complement activation is unclear. To explore the role of TLR9 in C3a expression, prostatic fibroblasts from wild type and TLR9-knockout mice were treated with CpG oligonucleotides, ODN 1826 (synthetic ligand for TLR9, CpG-ODN) or LNCaP-conditioned medium. TAK1 phosphorylation and C3a expression by LNCaP-CM was found to be dependent on TLR9 expression (FIG. 2A). DNase1 treatment of LNCaP-CM reduced TLR9 protein expression as well as C3a expression by wild type mouse fibroblasts. Testing prostatic fibroblasts generated from TLR9 knockout mice demonstrated no TAK1 activation or C3a expression under the same conditions. However, treating the prostatic fibroblasts with CpG-ODN generated dramatically less C3a compared to treatment with LNCaP-CM and was comparable to that found when LNCaP-CM was treated with DNase. ELISA studies corroborated TRAMPC2-CM and LNCaP-CM, but not CpG-ODN, triggered the release of C3a into the media of CAF at a significantly greater levels compared to NAF (FIG. 2B). These results showed LNCaP-CM induces TLR9 and C3a protein expression and this is inhibited by DNase treatment of the CM, indicating PCa-derived mtDNA can mediate paracrine signaling with CAF for the induction of TLR9 downstream signaling.

It is interesting to note that while CpG/mtDNA was sufficient to activate TLR9 downstream of DEC105 in CAF, only PCa epithelial CM was sufficient for the expression and secretion of C3a. Complement processing can occur through an enzymatic activation cascade or alternative pathways culminating in the cleavage of complement C3. Cleavage of C3 results in generation of C3a and C3b that is well described for microbe opsonization and activation of proinflammatory signaling. As the classical pathway involving the complex of complement proteins C1b and C2b for C3 cleavage was not likely in cultured fibroblasts, the alternative pathway involving reactive oxygen mediated cleavage was tested in CAF. Not surprisingly, the treatment of CAF with LNCaP-CM, resulted in reactive oxygen generation, as shown by DCFDA fluorescent quantitation by FACS analysis and visualized by fluorescent microscopy (FIG. 2C, D). CpG-ODN treatment promoted no such reactive oxygen signal and N-acetylcysteine (used as a reactive oxygen inhibitor) suppressed LNCaP-induced reactive oxygen as well as C3a generation. As catalase can mitigate the reactive oxygen content of cells, its activity was measured in CAF. We found that catalase activity in CAF was significantly suppressed by LNCaP-CM, compared to either untreated control or CpG-ODN treatment (FIG. 2E). Western blotting demonstrated N-acetylcysteine supplementation blocked the conversion of C3 to C3a induced by LNCaP-CM (FIG. 2F). Inhibition of catalase by 3-Amino-1,2,4-triazole had no effect on C3a generation when combined with CpG-ODN. Ultimately, both CpG-ODN and LNCaP-CM induced C3 expression in CAF, but C3a expression was dependent on the suppression of catalase activity and the induction of reactive oxygen by LNCaP-CM (FIG. 2G).

C3a signaling enhances PCa expansion. In an effort to determine the reciprocal epithelial response to anaphylatoxin C3a expressed by CAF, we tested the impact of established complement agonists and antagonists on PCa expansion. We found that LNCaP, PC3, and TRAMPC2 all expressed the anaphylatoxin C3a receptor (C3aR, FIG. 8A). In reinforcing the requirement of a paracrine TLR9-mediated anaphylatoxin C3a signaling axis, we found limited expression of DEC205, TLR9, and C3a by the three PCa epithelial lines, although HMGB1 was heterogeneously expressed (FIG. 8B). Next, the effect of C3a signaling on PCa cells was tested by incubating LNCaP, PC3, and TRAMPC2 with an agonist peptide of C3aR or scrambled peptide. Agonist peptides to C3aR were used rather than C3a itself because anaphylatoxins are extremely labile. Exposure of 0.1 µM C3aR agonist for 48 hours increased proliferation measured by Ki67 expression in LNCaP (28%), PC3 (30%) and TRAMPC2 (21%) compared to scrambled peptide treated cells (FIG. 8C). We further investigated the effect of C3aR agonist peptides on the PI3K/AKT signaling pathway in PCa cells and found an enhanced phosphorylation of AKT as a result of stimulation of C3aR (FIG. 3A). We also found that C3a strongly activated downstream MAP kinase signaling pathways by phosphorylation of p42/44 MAPK (p-ERK1/2). Up-regulation of Bcl-2 expression was identified as a downstream signaling molecule of AKT, in support of cell survival.

To validate the observation of C3a signaling, we allografted mouse prostatic fibroblasts with PCa epithelia into syngeneic C57B/6 mice. We grafted either wild type or TLR9-knockout fibroblasts and recombined them with luciferase-expressing TRAMPC2 cells under the renal capsule. After the tumors were visible by bioluminescent imaging, the mice were treated with either vehicle (control) or SB290157, a C3aR antagonist. Within 3 weeks of grafting, the tumors with wild type fibroblasts expanded reproducibly, however, the treatment with SB290157 had significantly smaller tumor size than the vehicle treated mice (FIG. 3B, C). Interestingly, the allografts with TLR9-knockout fibroblasts had negligible tumor growth, confirming the role of the associated paracrine signaling axis dependent on TLR9 and C3a. We were only able to perform immunohistochemistry on the grafts with wild type fibroblasts and TRAMPC2, as not enough tissue was available from grafts with TLR9-knockout fibroblasts. Tumor cell mitosis, as determined by phosphorylated-histone H3 expression was significantly lower when the host mice were treated with SB29157 (FIG. 3D). The activation of AKT, localized by phosphorylated AKT staining, was elevated in the tumor cells of mouse allografted with wild type fibroblasts and reversed in SB290157 treated mice. SB290157 was found to both significantly diminish tumor proliferation and elevate cell death, as localized by TUNEL staining.

Complement anaphylatoxins have a wide spectrum of proinflammatory effects. C3a is particularly regarded for the chemotaxis of mast cells, basophils and eosinophils. As T lymphocytes are confirmed regulators of tumor progression and known to respond to C3a, we measured the impact of C3a antagonism on T cell recruitment to the tumors. FACS analysis of the CD3+ T cells showed they were similarly recruited to the tumors regardless of C3a antagonist or fibroblast TLR9 status (FIG. 3E). However, CD8+ T cell activation, as determined by the expression of the costimulatory molecule CD69+, was appreciably downregulated by C3a antagonist and further downregulated in tumors with TLR9-knockout fibroblasts. These findings suggested that the recruitment of cytotoxic T cells is not likely the mediator of the tumor stromal-epithelial reciprocal TLR9/C3a signaling axis.

Synergistic effect of docetaxel and SB 290157 inhibit tumor expansion. Based on the observed activation of AKT by C3a in PCa epithelia, we were curious as to the role of this pro-survival signal in the context of a mediator of cell death, such as chemotherapy. To initially determine the clinical relevance of the TLR9/C3a signaling axis in PCa patients, we measured plasma mtDNA content in men pre-treatment and undergoing docetaxel treatment, in a paired fashion. Docetaxel induced a dramatic increase in circulating mtDNA in the PCa patients (P = 0.006, FIG. 4C). In parallel, mice treated with docetaxel (6 mg/kg/week) for three weeks showed significant elevation of plasma mtDNA content (P < 0.05, FIG. 4B). In examining the direct effect of docetaxel on PCa epithelia, we found docetaxel to profoundly elevate mtDNA secretion by LNCaP, PC3, and TRAMPC2 cells in a dose dependent manner (FIG. 4C). Of note, higher doses of docetaxel were used for PC3 cells compared to the other two lines due to its inherent resistance. In trying to determine why greater mtDNA secretion was associated with docetaxel treatment we revealed elevated LC3 activation, p62, and beclin expression in both cytoplasmic and mitochondrial subcellular fractions, suggesting both autophagy and mitophagy induction (FIG. 4D). Curiously, chemotherapy-induced cell death resulted in the release of mtDNA without its degradation. Docetaxel induction of endoplasmic reticulum (ER)-stress proteins, p62 and CHOP, concomitant of mitophagy, with beclin upregulation, in LNCaP and PC3 supported a means of mtDNA degradation escape (FIG. 4E). The influence of stromal-epithelial crosstalk in the development of docetaxel resistance was tested in co-cultured PC3 cells and CA within a 3-dimentional matrix of collagen I and Matrigel. EpCAM+/Ki67+ proliferative epithelia with PC3 and CAF co-culture was double that when PC3 cells were grown alone (FIG. 4F). Additional treatment with docetaxel did not appreciably reduce the CAF-induced proliferative epithelial fraction. The C3 antagonist, SB290157, restored the sensitivity of PC3 cells to docetaxel. Drug interaction studies reveled that low doses of SB290157 sensitized the otherwise resistant PC3 cells to docetaxel in a synergistic manner (fractional inhibitory concentration index < 0.5, FIG. 4G, FIG. 9A).

The therapeutic significance of the observed stromal-epithelial crosstalk was tested in male nude mice with tissue recombinant xenografts of CAF and PC3 cells. Tumor growth curves indicate tumor volume was not significantly reduced by treatment with the low dose of docetaxel (6 mg/kg/week) alone, compared to vehicle treatment (FIG. 5A). However, the combined treatment of docetaxel and SB290157 significantly limited tumor growth (P < 0.05). No significant impact on the body weight was observed in any of treatment groups, in support of a taxane therapy strategy with minimal toxicity (FIG. 9B). Western blotting of the tumor tissues revealed the activation of TAK1, AKT, and ERK1/2 in docetaxel-treated mice, compared to vehicle treatment (FIG. 5B). The upregulation of plasma mtDNA in docetaxel-treated mice supported the elevated phosphorylation of TAK. It was also observed SB290157 reduced AKT and ERK1/2 phosphorylation induced by docetaxel and downstream C3a was unaffected by SB290157. Importantly, Blc2 was reduced by SB290157. Histology of the tumors and immunohistochemistry of the corresponding tissues allowed us to localize and quantitate the relevant signaling molecules (FIG. 5C, FIG. 9C). The significant docetaxel-induction of phosphorylated-TAK1, C3, and TUNEL staining was not altered by C3 antagonism. However, docetaxel treatment induction of cell survival pathway and mitosis, as respectively quantitated by phosphorylation of AKT and phosphorylated histone-H3, was significantly diminished by the combined treatment of docetaxel and SB290157. Combination of SB290157 with low doses of docetaxel also resulted in limited tumor growth.

### Example 3

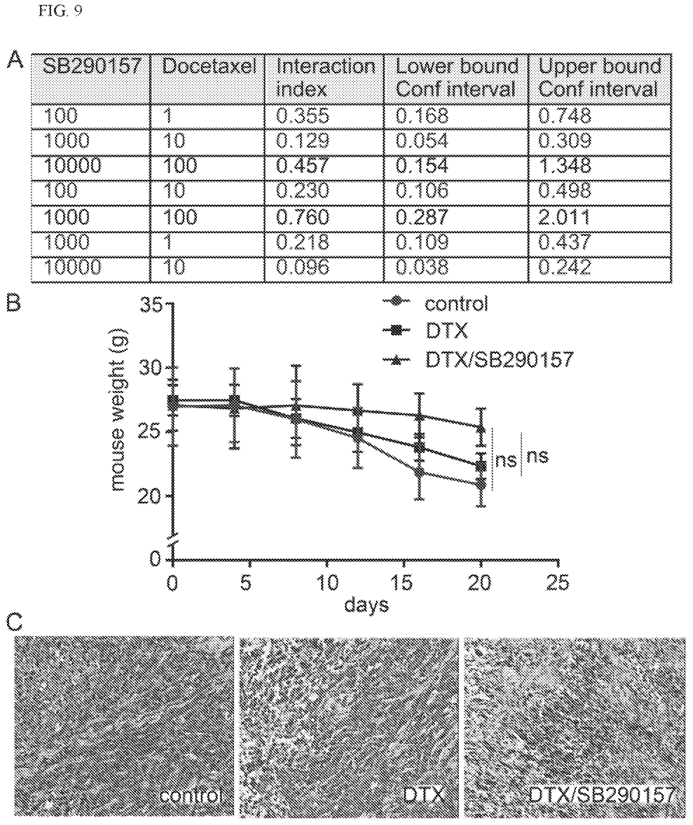

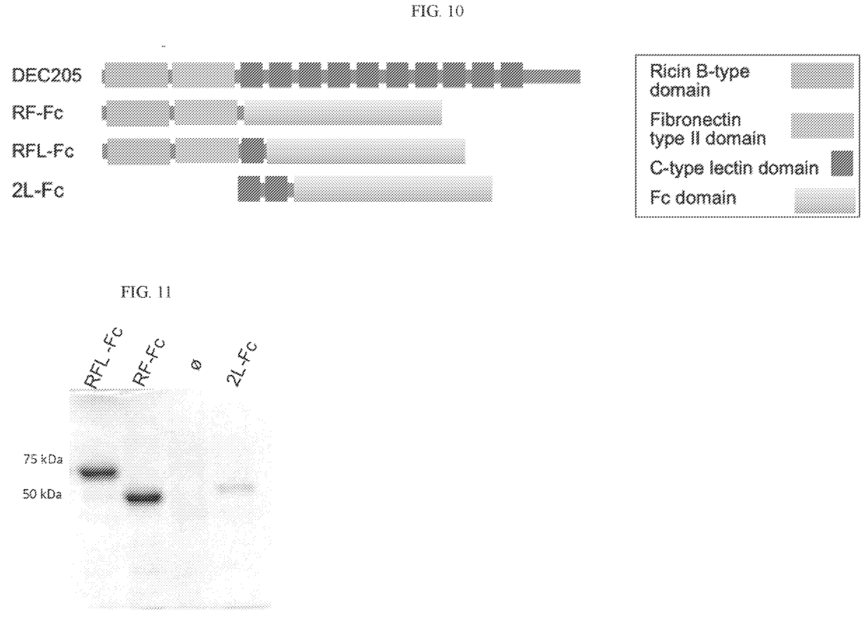

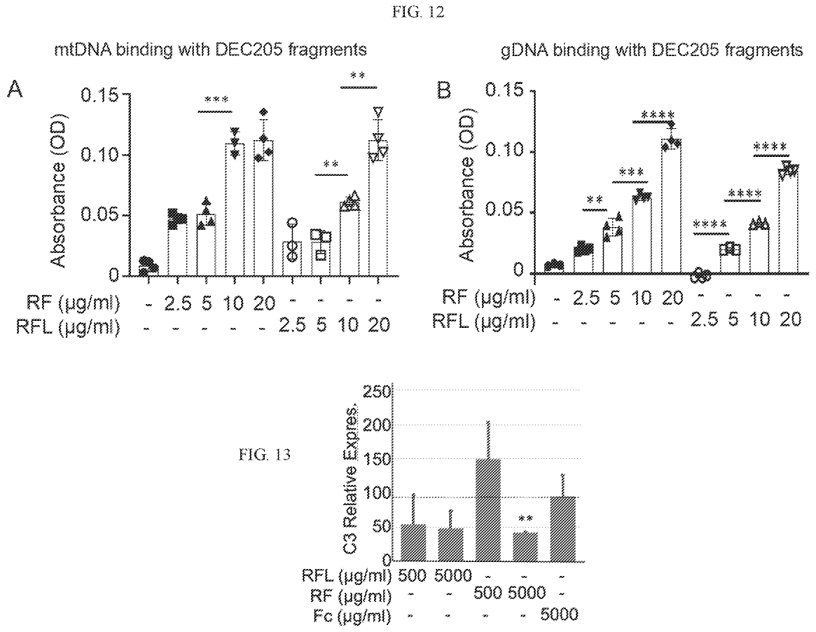

Based on the identification that DEC205 (LY75, CD205, DEC-205) binds mitochondrial DNA (mtDNA; PNAS 2020 11:8515), the protein domains responsible for mtDNA were identified (FIG. 10 and FIG. 11). ELISA designed by immobilizing mtDNA or genomic DNA (gDNA) to a 96-well plate and subsequent incubation with RF-Fc or RFL-Fc demonstrated binding of both DNA subtypes in a concentration dependent manner compared to Fc domain alone. The ricin type B lectin and fibronectin type II lectin domains (RF) conjugated to IgG1 Fc (RF-Fc) demonstrated a two-fold higher affinity for mtDNA over gDNA (FIG. 12). In comparison, the ricin type B lectin, fibronectin type II lectin domains, with a single C-type lectin domain (RFL) conjugated to IgG1 Fc (RFL-Fc) demonstrated similar affinity for mtDNA and gDNA. The remaining C-type lectin domains were found to bind both genomic DNA (gDNA) and mtDNA at similar affinity based on binding analysis of 2L-Fc and 6L-Fc (data not shown). The conjugation of the DEC205 domains to the antibody Fc domain allowed for superior protein folding, expression, and stability. The binding of RF-Fc and RFL-Fc to DNA was normalized to that of Fc binding at the same respective concentration. Each of the DEC205 domains were conjugated to a mouse IgG1 antibody Fc domain. The highly conserved human IgG1 antibody Fc domain can replace the mouse Fc domain for human therapeutic application.

The expression of complement C3 by cancer associated fibroblasts in response to mtDNA generated by cancer cells was demonstrated to mediate chemotherapy resistance in prostate cancer cells, namely docetaxel (PNAS 2020 11:8515). The depletion of mtDNA by RF-Fc significantly reduced C3 expression (FIG. 13).

### Example 4

### Application of the RF-Fc or RFL-Fc

1) The RF-Fc and RFL-Fc can be used for the detection of blood mtDNA content. Currently, DNA first needs to be extracted prior to a PCR-based assay for mtDNA content is required. Thus, a straightforward sandwich ELISA-based assay with RF-Fc for the detection of mtDNA. RFL-Fc can similarly be used for the detection of total DNA (gDNA and mtDNA) in circulation. The detection assay could include an ELISA similar to that demonstrated in FIG. 12, where the plasma from a subject is coated in a 96 well plate for subsequent incubation with RF-Fc or RFL-Fc that is directly conjugated to horseradish peroxidase (HRP) or via a secondary antibody strategy for development by a standard colorimetric peroxidase reaction. Other iterations employing the RF-Fc or RFL-Fc cold be where the Fc conjugates are immobilized on the plate prior to plasma incubation, washing and subsequent HRP-crosslinked RF-Fc for the detection of mtDNA for a sandwich ELISA technique. Similarly, HRP-crosslinked RFL-Fc can be used for gDNA detection. Alternatively, either RF-Fc or RFL-Fc can be immobilized on a bead as part of a bead-array in a multiplexing assay (e.g. Lumina box). Other direct immobilization techniques such as on a gold substrate can enable a change in impedance. Circulating cell-free DNA is a mediator of systemic inflammation. Elevated circulating mtDNA are found in patients with cancer, trauma, infections, stroke, autoimmune, cachexia, and cardiac disease. Lupus patients are diagnosed by the detection of circulating cell free DNA. There are many triggers of cell-free mtDNA secretion inclusive of inflammatory cytokines and therapeutics used in cancer patients (e.g. docetaxel, cisplatin, doxorubicin, and androgen targeted therapy). A facile detection of mtDNA or gDNA in circulation could identify subjects that may require DNA depletion therapy.

2) RF-Fc and RFL-Fc can be used to deplete circulating mtDNA/gDNA to sensitize tumors to chemotherapy.

i) Direct injection intravenously. The introduction of RF-Fc or RFL-Fc into individual with elevated circulating mtDNA or gDNA can be used to deplete the antigen by way of Fc-gamma receptors found on liver endothelial cells. The captured mtDNA or gDNA would then be excreted through the stool.

The Fc conjugates can be given at a dosing schedule as follows for 28 days during the time of chemotherapy treatment: split dose regimen (Max weight for dose calculation = 85 kg):
for cycle 1, days 1 and 4: initial doses: 3 mg/kg IV for one dose on day 1, followed by 7 mg/kg on day 4, followed by full dose regimen: 10 mg/kg IV for one dose on days 8, 15 and 22.
cycle 2 onwards: 10 mg/kg IV for one dose on days 1, 8, 15 and 22

ii) Immobilize RF-Fc or RFL-Fc to dextran beads or Sepharose beads or other solid support for passing blood through a blood filtration system that can selectively remove mtDNA/gDNA from circulating blood. Blood would enter through medical tubing of the devices, the filtration chamber would be exposed to the subject’s blood, providing an opportunity to capture the DNA. The blood would then be restored back into the individual. Such a process can be used prior to chemotherapy infusion cycles for a cancer patient.

iii) The RF-Fc or RFL-Fc proteins that are expressed by Bacillus subtilis (or other enteric bacteria such as Lactococus lactis) and introduced into the gut microbiome by ingestion. The colonization of the enteric bacteria can be done prior to chemotherapy treatment. Chemotherapy is known to cause “leaky gut,” disintegration of the tight-junctions of colonic epithelia, separating the contents of the colon and circulation. Chemotherapy sensitization can be enabled by the introduction of RF-Fc or RFL-Fc in circulation and depletion of mtDNA/gDNA by liver Fc-gamma receptor for excretion. Enteric bacteria inclusive of, Bacillus subtilis and Lactococus lactis are known to improve gut health.

Applications for RF-Fc or RFL-Fc is inclusive of cancer patients, cachexic patients are also known to have elevated mtDNA in circulation associated with toll like receptor mediated inflammation that causes muscle wasting. The depletion of mtDNA in cachexic patients can limit muscle wasting. Similarly, lupus patients are recognized to have gDNA in circulation associated with disease inflammation. RFL-Fc can be used for lupus subjects.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

1. A protein, comprising:
a polypeptide that binds to mitochondrial DNA (mtDNA), genomic DNA (gDNA), or both; and
a Fc fragment of IgG receptor gamma (FcgRIIb) or a fragment thereof.

2. The protein of claim 1, wherein the polypeptide that binds to mtDNA, gDNA or both comprises a fragment of DEC205 or a fragment of DEC205 with one or more amino acid deletions, additions or substitutions.

3. The protein of claim 1, wherein the fragment of DEC205 is a polypeptide at least 90% identical to at least one domain selected from the group consisting of Ricin B-type lectin domain, fibronectin type II lectin domain, and at least one C-type lectin domain.

4. The protein of claim 1, wherein the fragment of DEC205 is a polypeptide at least 90% identical to at least two domains selected from the group consisting of Ricin B-type lectin domain, fibronectin type II lectin domain, and at least one C-type lectin domain.

5. The protein of claim 1, wherein the fragment of DEC205 is a polypeptide at least 90% identical to at least three domains selected from the group consisting of Ricin B-type lectin domain, fibronectin type II lectin domain, and at least one C-type lectin domain.

6. The protein of claim 1, wherein the fragment of DEC205 is a polypeptide at least 90% identical to Ricin B-type lectin domain, fibronectin type II lectin domain, or both.

7. The protein of claim 1, wherein the fragment of DEC205 is a polypeptide at least 90% identical to Ricin B-type lectin domain and fibronectin type II lectin domain.

8. The protein of claim 1, wherein the fragment of DEC205 is a polypeptide at least 90% identical to Ricin B-type lectin domain, fibronectin type II lectin domain, and at least one C-type lectin domain.

9. The protein of claim 1, wherein the fragment of DEC205 is a polypeptide at least 90% identical to at least one C-type lectin domain.

10. The protein of claim 1, wherein the fragment of DEC205 is a polypeptide at least 90% identical to at least two C-type lectin domains.

11. The protein of claim 1, wherein the fragment of DEC205 comprises a polypeptide is at least 90% identical to a sequence selected from the group consisting of SEQ ID NO: 1, SEQ ID NO:2, and SEQ ID NO:3.

12. The protein of claim 1, wherein the fragment of DEC205 comprises a polypeptide that has a sequence selected from the group consisting of SEQ ID NO: 1, SEQ ID NO:2, and SEQ ID NO:3.

13. The protein of claim 1, wherein the fragment of DEC205 comprises a polypeptide is at least 90% identical to a sequence comprising SEQ ID NO:4.

14. The protein of claim 1, wherein the fragment of DEC205 comprises a polypeptide having at least 168 consecutive amino acids of SEQ ID NO:4.

15. The protein of claim 1, wherein the fragment of DEC205 comprises a polypeptide having 168 to 414 consecutive amino acids of SEQ ID NO:4.

16. The protein of claim 1, wherein the fragment of DEC205 comprises a polypeptide having 183 to 368 consecutive amino acids of SEQ ID NO:4.

17. The protein of claim 1, wherein the fragment of DEC205 comprises a polypeptide having 202 to 322 consecutive amino acids of SEQ ID NO:4.

18. The protein of claim 1, wherein the fragment of DEC205 comprises a polypeptide having 220 to 276 consecutive amino acids of SEQ ID NO:4.

19. The protein of claim 1, wherein the Fc fragment of IgG receptor gamma (FcgRIIb) comprises a human IgG1 Fc domain or a human IgG1 Fc domain with up to 22 amino acid additions, deletions, and/or substitutions.

20. The protein of claim 1, wherein the Fc fragment of IgG receptor gamma (FcgRIIb) or the fragment thereof comprises at least 205 consecutive amino acids as set forth in SEQ ID NO: 5.

21. The protein of claim 1, wherein the Fc fragment of IgG receptor gamma (FcgRIIb) or the fragment thereof comprises a sequence with at least 90% sequence identity with SEQ ID NO: 5.

22. The protein of claim 1, wherein the Fc fragment of IgG receptor gamma (FcgRIIb) comprises a polypeptide having the sequence as set forth in SEQ ID NO: 5.

23. The protein of claim 1, wherein the Fc fragment of IgG receptor gamma (FcgRIIb) is a mouse IgG1 Fc domain, or a mouse IgG1 Fc domain with up to 21 amino acid additions, deletions, and/or substitutions.

24. The protein of claim 1, wherein the Fc fragment of IgG receptor gamma (FcgRIIb) or the fragment thereof comprises at least 209 consecutive amino acids as set forth in SEQ ID NO:6.

25. The protein of claim 1, wherein the Fc fragment of IgG receptor gamma (FcgRIIb) or the fragment thereof comprises a sequence with at least 90% sequence identity with SEQ ID NO:6.

26. The protein of claim 1, wherein the Fc fragment of IgG receptor gamma (FcgRIIb) comprises a polypeptide having the sequence as set forth in SEQ ID NO:6.

27. The protein of claim 1, further comprising a signal sequence, a linker, or both.

28. The protein of claim 27, wherein the signal sequence comprises the amino acids as set forth in SEQ ID NO:7.

29. The protein of claim 1, wherein the protein is selected from a protein having the sequence as set forth in any one of amino acids 24-435 of SEQ ID NO:8, amino acids 24-583 of SEQ ID NO:9, amino acids 24-529 of SEQ ID NO:10, amino acids 24-440 of SEQ ID NO: 11, amino acids 24-588 of SEQ ID NO: 12, or amino acids 24-534 of SEQ ID NO: 13.

30. The protein of claim 1, wherein the protein is selected from a protein comprising the sequence of
SEQ ID NO:1 and SEQ ID NO: 5; or
SEQ ID NO:2 and SEQ ID NO: 5; or
SEQ ID NO:3 and SEQ ID NO: 5; or
SEQ ID NO:1 and SEQ ID NO:6; or
SEQ ID NO:2 and SEQ ID NO:6; or
SEQ ID NO:3 and SEQ ID NO:6.

31. The protein of claim 1, wherein the protein is selected from a protein having the sequence as set forth in any one of SEQ ID NO:8, SEQ ID NO:9, SEQ ID NO:10, SEQ ID NO:11, SEQ ID NO:12, or SEQ ID NO: 13.

32. The protein of claim 1, wherein the polypeptide that binds to mtDNA, gDNA or both comprises a fragment of toll-like receptor 9 (TLR9) or a fragment of TLR9 with one or more amino acid deletions, additions or substitutions.

33. The protein of claim 1, further comprising an Fc region of an antibody or a fragment thereof.

34. The protein of claim 1, capable of depleting circulating mtDNA.

35. The protein of claim 1, capable of depleting circulating genomic DNA (gDNA).

36. A nucleic acid encoding a protein of claim 1.

37. A cell producing a protein of claim 1.

38. The cell of claim 37, wherein the cell is a bacterial cell, a Chinese hamster ovarian cell (CHO) or a baby hamster kidney cell (BHK).

39. The cell of claim 38, wherein the bacterial cell is Bacillus subtilis or Lactococus lactis.

40. A combination, comprising:
a protein of any of claim 1; and
a therapeutic agent.

41. (canceled)

42. (canceled)

43. (canceled)

44. (canceled)

45. (canceled)

46. (canceled)

47. (canceled)

48. A device, comprising:
at least one inlet;
at least one outlet;
at least one chamber comprising a solid substrate; and
a protein of claim 1, immobilized on the solid substrate.

49. (canceled)

50. (canceled)

51. (canceled)

52. (canceled)

53. (canceled)

54. (canceled)

55. (canceled)

56. (canceled)

57. A method of reducing circulating mitochondrial DNA (mtDNA), genomic DNA (gDNA), or both in a mammalian subject, comprising:
administering a protein of claim 1 to the mammalian subject.

58. (canceled)

59. (canceled)

60. (canceled)

61. (canceled)

62. (canceled)

63. A method of measuring circulating mitochondrial DNA (mtDNA), genomic DNA, or both comprising:
obtaining a biological sample; and
contacting a protein of claim 1 to the biological sample.

64. (canceled)

65. (canceled)

66. (canceled)

67. (canceled)
